SEQUENCE LISTING (33 sequences)

SEQ ID NO: 1 - DNA, 1918 bp, synthetic construct
  source 1..1918: mol_type = other DNA
gtagaaacaccttgtgatcaccctggcagtgattatgagcttcaggtctggaatcagactgctggctagactaatcagactggttagaatccaggatttatcatgtgtcaattgtgtgacttttggaaagtagattaattcatgaacaccatttcctcctctgaagtgaggaataataaccgtgcttttctcacctcaggggcagatgctattttttaggcaagatctgcttagaggtcccagtttcttattgctgcccttctctgctgtaactcttctcccctcatagacagctccactcctccagcctgctgcttgttgacaccaattctctggaaggggagtgacatcagtcatatatgctttaggggggtatttaagctgctatgactcttctcaggggcatttctctccaaagtctcacttctaatcaccaggccacctgctaatgataattagatcatgggtggtcagatgaaggaggcactgggagaggggaaatccccatatctctggtatcccaggaaatagataaccatcattccagccatccttttgttttctttctttctttctttctttcttactttctttctttctttctttccttctctctttcttccttccgctctctctctgtcaaccaggctggagtgcagtggcgtgatctcagctcactgcaacctccacctcctgggttcaagtgattctccttcctcagcctcccgagtagctgggactacaggcgcctgccaccatgcccagctaatttttggtaattttagtagagacggggtttcaccgtggtctcgatctcctgacctcgtgatccgaccgcctcggcctctcaaagtgctgggattacaggcgtgaaccaccatgcccagcctatccttttgttttccatcctgtgttggcttggtgggggagaggaggtgttgacacgtggaggacacacatataaggcattcttgggtgacttcgtcatcactggaccctatctctcaaaattccagcgaaatctgctcttccctttaaggagtgaaagaagggtcagcattccagaagttcctggtcatacccaggcttttaatgaattgccactggggaatcagcatcccgttgctgtaaggactataagatggcggattgtgagagcatagggaaaggtctcggaggtctcttgtccttgctccacgcaggtctttctggcctgaaaatcccgttgaagagagcagctcttgagagtttgctccaagttccctcggggtgatcagcaccacggacaggggccagggcgcccccgaggacccgcaggcaggcagggtgcacagcggggagcaggtgctgcgctacgtgcgggccagggaactcgcgcggggaggggagaggcgccgcgggtggcggggtcttggccggggctgttttcgctgtgagatcaccccgtgctcccagcgctcacgtcggtcctcggaaagccggggtcctccctgccttttccagcaacggtggggtggggaggcaggaagaaagcgccaacctaggaccccggagatttgcagcaaaggaagaagcgggagacgggcacttgtctgtgtctccagcgcgttcctgccccccgccgacccggcccatttctatacaaggtcgctctgcccggtctccacctcccacgtgcaggccgcggaggggctcattcccgggccctgatctcagaggcccggaatgtggctgataaatcagagactagacctgcatggcaggcaggcccgacactcagctccaggataaaaggccacggtgtcccgaggagccaggaggagcaccccgcaggctgagggcaggtgggaagcaaacccggacgcatcgcagcagcagcagcagcagcagaagcagcagcagcagcctccgcagtccc

SEQ ID NO: 2 - DNA, 126 bp, synthetic construct
  source 1..126: mol_type = other DNA
atgcaggcggctcggatggccgcgagcttggggcggcagctgctgaggctcgggggcggaagctcgcggctcacggcgctcctggggcagccccggcccggccctgcccggcggccctatgccggg

SEQ ID NO: 3 - protein, 28 aa, synthetic construct
MAASPHTLSSRLLTGCVGGSVWYLERRT

SEQ ID NO: 4 - protein, 122 aa, synthetic construct
MAFKSFIYSKGYHRSAAQKKTATSFFDSSYQYLRQNQGLVNSDPVLHASHLHPHPVVVANVNYNNVDDILHPHDLDSSINNTNNPLTHEELLYNQNVSLRSLKQQQSTNYVNNNNNNQHRYY

SEQ ID NO: 5 - protein, 174 aa, synthetic construct
MRKRSLRCHLWSANASLSPRKDEVTSRKESENLVKGKKNKKSHLHLLLFTASKIGTDSVFDVQKSKECCKELGLLFTSLIHSIGSFPFDEEPKAAAVFLPGSLPQLTVLVLAPGSGSCPTGKSTPHLAASGRNAELLRPQNSMIVRQFTCRGTISSHLCAHLRKPHDSRNMARP

SEQ ID NO: 6 - protein, 56 aa, synthetic construct
MLRRTSFNFTGRAMISRGSPEWSHRLDLKKGKKTTMMHKLGTSKPNNALQYAQMTL

SEQ ID NO: 7 - protein, 46 aa, synthetic construct
MISRSALSRGSQLALRRPAAAKTAQRGFAAAAASPAASYEPTTIAG

SEQ ID NO: 8 - protein, 128 aa, synthetic construct
MPELILYVAITLSVAERLVGPGHACAEPSFRSSRCSAPLCLLCSGSSSPATAPHPLKMFACSKFVSTPSLVKSTSQLLSRPLSAVVLKRPEILTDESLSSLAVSCPLTSLVSSRSFQTSAISRDIDTA

SEQ ID NO: 9 - protein, 115 aa, synthetic construct
MYRLMSAVTARAAAPGGLASSCGRRGVHQRAGLPPLGHGWVGGLGLGLGLALGVKLAGGLRGAAPAQSPAAPDPEASPLAEPPQEQSLAPWSPQTPAPPCSRCFARAIESSRDLL

SEQ ID NO: 10 - protein, 140 aa, synthetic construct
MTVLAPLRRLHTRAAFSSYGREIALQKRFLNLNSCSAVRRYGTGFSNNLRIKKLKNAFGVVRANSTKSTSTVTTASPIKYDSSFVGKTGGEIFHDMMLKHNVKHVFGYPGGAILPVFDAIYRSPHFEFILPRHEQAAGHA

SEQ ID NO: 11 - protein, 125 aa, synthetic construct
MILCPLEAFIVQHILTISVMGLLSCFRSTVLRKCSKGSSGMSRFLYTNNFQRNLISSGGNESYYGYFNRRSYTSLYMGTGTVGGITSARIRVPNVGCEGFMCSSHLSITQRNSRLIHSTSKIVPN

SEQ ID NO: 12 - protein, 107 aa, synthetic construct
MALQQAAPRVFGLLGRAPVALGQSGILTGSSGFKNQGFNGSLQSVENHVYAQAFSTSSQEEQAAPSIQGASGMKLPGMAGSMLLGKSRSGLRTGSMVPFAAQQAMNM

SEQ ID NO: 13 - protein, 87 aa, synthetic construct
MWRLRRAAVACEVCQSLVKHSSGIKGSLPLQKLHLVSRSIYHSHHPTLKLQRPQLRTSFQQFSSLTNLPLRKLKFSPIKYGYQPRRN

SEQ ID NO: 14 - protein, 56 aa, synthetic construct
MAVLWRLSAVCGALGGRALLLRTPVVRPAHISAFLQDRPIPEWCGVQHIHLSPSHH

SEQ ID NO: 15 - protein, 162 aa, synthetic construct
MAAILGDTIMVAKGLVKLTQAAVETHLQHLGIGGELIMAARALQSTAVEQIGMFLGKVQGQDKHEEYFAENFGGPEGEFHFSVPHAAGASTDFSSASAPDQSAPPSLGHAHSEGPAPAYVASGPFREAGFPGQASSPLGRANGRLFANPRDSFSAMGFQRRF

SEQ ID NO: 16 - protein, 117 aa, synthetic construct
MALLLRHSPKLRRAHAILGCERGTVVRHFSSSTCSSLVKEDTVSSSNLHPEYAKKIGGSDFSHDRQSGKELQNFKVSPQEASRASNFMRASKYGMPITANGVHSLFSCGQVVPSRCF

SEQ ID NO: 17 - protein, 66 aa, synthetic construct
MASTRVLASRLASQMAASAKVARPAVRVAQVSKRTIQTGSPLQTLKRTQMTSIVNATTRQAFQKRA

SEQ ID NO: 18 - protein, 44 aa, synthetic construct
MLAARLVCLRTLPSRVFHPAFTKASPVVKNSITKNQWLLTPSRE

SEQ ID NO: 19 - protein, 68 aa, synthetic construct
MASRVLSAYVSRLPAAFAPLPRVRMLAVARPLSTALCSAGTQTRLGTLQPALVLAQVPGRVTQLCRQY

SEQ ID NO: 20 - protein, 67 aa, synthetic construct
MFACAKLACTPSLIRAGSRVAYRPISASVLSRPEASRTGEGSTVFNGAQNGVSQLIQREFQTSAISR

SEQ ID NO: 21 - protein, 273 aa, synthetic construct
MALQQAAPRVFGLLGRAPVALGQSGILTGSSGFKNQGFNGSLQSVENHVYAQAFSTSSQEEQAAPSIQGASGMKLPGMAGSMLLGKSRSGLRTGSMVPFAAQQAMNMGGMAAILGDTIMVAKGLVKLTQAAVETHLQHLGIGGELIMAARALQSTAVEQIGMFLGKVQGQDKHEEYFAENFGGPEGEFHFSVPHAAGASTDFSSASAPDQSAPPSLGHAHSEGPAPAYVASGPFREAGFPGQASSPLGRANGRLFANPRDSFSAMGFQRRFGG

SEQ ID NO: 22 - protein, 132 aa, synthetic construct
MASTRVLASRLASQMAASAKVARPAVRVAQVSKRTIQTGSPLQTLKRTQMTSIVNATTRQAFQKRAMASTRVLASRLASQMAASAKVARPAVRVAQVSKRTIQTGSPLQTLKRTQMTSIVNATTRQAFQKRA

SEQ ID NO: 23 - protein, 119 aa, synthetic construct
MALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPY

SEQ ID NO: 24 - protein, 391 aa, synthetic construct
MASTRVLASRLASQMAASAKVARPAVRVAQVSKRTIQTGSPLQTLKRTQMTSIVNATTRQAFQKRAMALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPYMTVLAPLRRLHTRAAFSSYGREIALQKRFLNLNSCSAVRRYGTGFSNNLRIKKLKNAFGVVRANSTKSTSTVTTASPIKYDSSFVGKTGGEIFHDMMLKHNVKHVFGYPGGAILPVFDAIYRSPHFEFILPRHEQAAGHAMASTRVLASRLASQMAASAKVARPAVRVAQVSKRTIQTGSPLQTLKRTQMTSIVNATTRQAFQKRA

SEQ ID NO: 25 - protein, 455 aa, synthetic construct
MALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPYMAAILGDTIMVAKGLVKLTQAAVETHLQHLGIGGELIMAARALQSTAVEQIGMFLGKVQGQDKHEEYFAENFGGPEGEFHFSVPHAAGASTDFSSASAPDQSAPPSLGHAHSEGPAPAYVASGPFREAGFPGQASSPLGRANGRLFANPRDSFSAMGFQRRFMALQQAAPRVFGLLGRAPVALGQSGILTGSSGFKNQGFNGSLQSVENHVYAQAFSTSSQEEQAAPSIQGASGMKLPGMAGSMLLGKSRSGLRTGSMVPFAAQQAMNMMFACAKLACTPSLIRAGSRVAYRPISASVLSRPEASRTGEGSTVFNGAQNGVSQLIQREFQTSAISR

SEQ ID NO: 26 - protein, 348 aa, synthetic construct
MALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPYMAAILGDTIMVAKGLVKLTQAAVETHLQHLGIGGELIMAARALQSTAVEQIGMFLGKVQGQDKHEEYFAENFGGPEGEFHFSVPHAAGASTDFSSASAPDQSAPPSLGHAHSEGPAPAYVASGPFREAGFPGQASSPLGRANGRLFANPRDSFSAMGFQRRFMFACAKLACTPSLIRAGSRVAYRPISASVLSRPEASRTGEGSTVFNGAQNGVSQLIQREFQTSAISR

SEQ ID NO: 27 - protein, 185 aa, synthetic construct
MASTRVLASRLASQMAASAKVARPAVRVAQVSKRTIQTGSPLQTLKRTQMTSIVNATTRQAFQKRAMALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPY

SEQ ID NO: 28 - protein, 455 aa, synthetic construct
MAAILGDTIMVAKGLVKLTQAAVETHLQHLGIGGELIMAARALQSTAVEQIGMFLGKVQGQDKHEEYFAENFGGPEGEFHFSVPHAAGASTDFSSASAPDQSAPPSLGHAHSEGPAPAYVASGPFREAGFPGQASSPLGRANGRLFANPRDSFSAMGFQRRFMALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPYMALQQAAPRVFGLLGRAPVALGQSGILTGSSGFKNQGFNGSLQSVENHVYAQAFSTSSQEEQAAPSIQGASGMKLPGMAGSMLLGKSRSGLRTGSMVPFAAQQAMNMMFACAKLACTPSLIRAGSRVAYRPISASVLSRPEASRTGEGSTVFNGAQNGVSQLIQREFQTSAISR

SEQ ID NO: 29 - protein, 455 aa, synthetic construct
MALQQAAPRVFGLLGRAPVALGQSGILTGSSGFKNQGFNGSLQSVENHVYAQAFSTSSQEEQAAPSIQGASGMKLPGMAGSMLLGKSRSGLRTGSMVPFAAQQAMNMMAAILGDTIMVAKGLVKLTQAAVETHLQHLGIGGELIMAARALQSTAVEQIGMFLGKVQGQDKHEEYFAENFGGPEGEFHFSVPHAAGASTDFSSASAPDQSAPPSLGHAHSEGPAPAYVASGPFREAGFPGQASSPLGRANGRLFANPRDSFSAMGFQRRFMALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPYMFACAKLACTPSLIRAGSRVAYRPISASVLSRPEASRTGEGSTVFNGAQNGVSQLIQREFQTSAISR

SEQ ID NO: 30 - protein, 285 aa, synthetic construct
MAAILGDTIMVAKGLVKLTQAAVETHLQHLGIGGELIMAARALQSTAVEQIGMFLGKVQGQDKHEEYFAENFGGPEGEFHFSVPHAAGASTDFSSASAPDQSAPPSLGHAHSEGPAPAYVASGPFREAGFPGQASSPLGRANGRLFANPRDSFSAMGFQRRFGGMALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPYGG

SEQ ID NO: 31 - protein, 394 aa, synthetic construct
MAAILGDTIMVAKGLVKLTQAAVETHLQHLGIGGELIMAARALQSTAVEQIGMFLGKVQGQDKHEEYFAENFGGPEGEFHFSVPHAAGASTDFSSASAPDQSAPPSLGHAHSEGPAPAYVASGPFREAGFPGQASSPLGRANGRLFANPRDSFSAMGFQRRFGGMALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPYGGMALQQAAPRVFGLLGRAPVALGQSGILTGSSGFKNQGFNGSLQSVENHVYAQAFSTSSQEEQAAPSIQGASGMKLPGMAGSMLLGKSRSGLRTGSMVPFAAQQAMNMGG

SEQ ID NO: 32 - protein, 574 aa, synthetic construct
MASTRVLASRLASQMAASAKVARPAVRVAQVSKRTIQTGSPLQTLKRTQMTSIVNATTRQAFQKRAMALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPYMTVLAPLRRLHTRAAFSSYGREIALQKRFLNLNSCSAVRRYGTGFSNNLRIKKLKNAFGVVRANSTKSTSTVTTASPIKYDSSFVGKTGGEIFHDMMLKHNVKHVFGYPGGAILPVFDAIYRSPHFEFILPRHEQAAGHAVSGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEMASTRVLASRLASQMAASAKVARPAVRVAQVSKRTIQTGSPLQTLKRTQMTSIVNATTRQAFQKRA

SEQ ID NO: 33 - protein, 810 aa, synthetic construct
MASTRVLASRLASQMAASAKVARPAVRVAQVSKRTIQTGSPLQTLKRTQMTSIVNATTRQAFQKRAMALLRAAVSELRRRGRGALTPLPALSSLLSSLSPRSPASTRPEPNNPHADRRHVIALRRCPPLPASAVLAPELLHARGLLPRHWSHASPLSTSSSSSRPADKAQLTWVDKWIPEAARPYMTVLAPLRRLHTRAAFSSYGREIALQKRFLNLNSCSAVRRYGTGFSNNLRIKKLKNAFGVVRANSTKSTSTVTTASPIKYDSSFVGKTGGEIFHDMMLKHNVKHVFGYPGGAILPVFDAIYRSPHFEFILPRHEQAAGHAMRKRSLRCHLWSANASLSPRKDEVTSRKESENLVKGKKNKKSHLHLLLFTASKIGTDSVFDVQKSKECCKELGLLFTSLIHSIGSFPFDEEPKAAAVFLPGSLPQLTVLVLAPGSGSCPTGKSTPHLAASGRNAELLRPQNSMIVRQFTCRGTISSHLCAHLRKPHDSRNMARPMALLLRHSPKLRRAHAILGCERGTVVRHFSSSTCSSLVKEDTVSSSNLHPEYAKKIGGSDFSHDRQSGKELQNFKVSPQEASRASNFMRASKYGMPITANGVHSLFSCGQVVPSRCFMPELILYVAITLSVAERLVGPGHACAEPSFRSSRCSAPLCLLCSGSSSPATAPHPLKMFACSKFVSTPSLVKSTSQLLSRPLSAVVLKRPEILTDESLSSLAVSCPLTSLVSSRSFQTSAISRDIDTAMASTRVLASRLASQMAASAKVARPAVRVAQVSKRTIQTGSPLQTLKRTQMTSIVNATTRQAFQKRA

(19) United States
(12) Patent Application Publication
(10) Pub. No.: US 2024/0189455 A1
(43) Pub. Date: Jun. 13, 2024

(54) COMPOSITIONS AND METHODS FOR TREATING RIGHT VENTRICLE DYSFUNCTION

(71) Applicant: The United States Government As Represented By The Department Of Veterans Affairs, Washington, DC (US)

(72) Inventors: Gaurav Choudhary, Providence, RI (US); Peng Zhang, Providence, RI (US)

(21) Appl. No.: 18/533,784

(22) Filed: Dec. 8, 2023

Related U.S. Application Data
(60) Provisional application No. 63/431,492, filed on Dec. 9, 2022.

(51) Int. Cl. A61K 48/00; C12N 9/10; C12N 9/12; C12N 15/86

(52) U.S. Cl. CPC A61K 48/005; A61K 48/0075; C12N 9/1029; C12N 9/12; C12N 15/86; C12N 2750/14143; C12N 2840/007; C12Y 203/01016; C12Y 207/10002

(57) ABSTRACT

Disclosed are nucleic acid sequences comprising a cardiomyocyte-specific, cardiac stress-induced promoter operably linked to a mitochondrial targeting sequence (MTS) and/or a gene of interest. Disclosed are vectors comprising one or more of the disclosed nucleic acids. Disclosed are methods of using the disclosed nucleic acid sequences or vectors for treating a subject in need thereof. Disclosed are methods of treating pulmonary hypertension (PH) in a subject comprising administering a therapeutically effective amount of a vector to the subject, wherein the vector comprises a nucleic acid sequence comprising a cardiomyocyte-specific, cardiac stress-induced promoter operably linked to a mitochondrial targeting sequence (MTS) and/or a gene that encodes a PH therapeutic, wherein the PH therapeutic is expressed in cardiomyocytes undergoing cardiac stress.

### CROSS-REFERENCE TO RELATED APPLICATIONS

This application claims the benefit of U.S. Provisional Patent Application No. 63/431,492, filed Dec. 9, 2022, each of which is incorporated by reference herein in its entirety.

### STATEMENT REGARDING FEDERALLY SPONSORED RESEARCH

This invention was made with government support under Grant Number 5I01CX001892-04 awarded by the Department of Veterans Affairs and 5R01HL148727-04 awarded by the National Institutes of Health. The government has certain rights in this invention.

### REFERENCE TO SEQUENCE LISTING

The Sequence Listing submitted Jan. 31, 2024 as an xml file named “37759.0464U2.xml,” created on Jan. 25, 2024, and having a size of 37,837 bytes is hereby incorporated by reference pursuant to 37 C.F.R. § 1.52(e)(5).

### BACKGROUND

Pulmonary hypertension (PH) is a progressive disorder characterized by high blood pressure in pulmonary arteries and is associated with pathological remodeling of the pulmonary vasculature. Despite better outcomes in the modern management era, observed mortality rates in PH patients remain high. Right ventricular (RV) dysfunction/failure consequent to high RV afterload is associated with high morbidity and mortality in PH. As the major cell type, cardiomyocytes play a central role in RV remodeling and dysfunction in PH. Mitochondrial dysfunction including energy deficiency, oxidative stress, and altered dynamics has been implicated in RV dysfunction and failure in PH. Thus, targeting impaired RV mitochondria in diseased RV cardiomyocytes in settings of PH may provide novel therapeutic strategies to improve RV function and subsequently improve prognosis in PH. To date, however, there are no available approaches to specifically manipulate gene expression in mitochondria of diseased RV cardiomyocytes in PH. The current approaches for gene manipulation in the heart in vivo target to both left and right ventricles, to all the cardiomyocytes (both diseased and non-stressed), and to whole cell levels (i.e., not to subcellular compartments such as mitochondria). This disclosed invention is aimed to fill this gap with mitochondrial targeted gene manipulation in diseased RV cardiomyocytes in PH, which is expected to be a powerful tool over prior technological approaches and can be used not only to advances our understanding in mechanistic investigations of RV dysfunction/failure in PH but also to open the opportunity for future translational therapeutics.

### BRIEF SUMMARY

Disclosed are nucleic acid sequences comprising a cardiomyocyte-specific, cardiac stress-induced promoter operably linked to a mitochondrial targeting sequence (MTS) and a gene of interest. Disclosed are nucleic acid sequences comprising a cardiomyocyte-specific, cardiac stress-induced promoter operably linked to a mitochondrial targeting sequence (MTS). Disclosed are nucleic acid sequences comprising a cardiomyocyte-specific, cardiac stress-induced promoter operably linked to a gene of interest. In some aspects, the cardiomyocyte-specific, cardiac stress-induced promoter is a human pro-B-type natriuretic protein (hBNP) promoter.

Disclosed are nucleic acid sequences comprising a human pro-B-type natriuretic protein (hBNP) promoter operably linked to a MTS and a gene of interest.

Disclosed are vectors comprising any of the nucleic acid sequences disclosed herein.

Disclosed are methods of treating a subject in need thereof with one or more of the disclosed nucleic acid sequences or vectors.

Disclosed are methods of treating pulmonary hypertension (PH) in a subject comprising administering a therapeutically effective amount of a vector to the subject, wherein the vector comprises a nucleic acid sequence comprising a cardiomyocyte-specific, cardiac stress-induced promoter operably linked to a MTS and/or a gene that encodes a PH therapeutic, wherein the PH therapeutic is expressed in cardiomyocytes undergoing cardiac stress.

Disclosed are methods of treating right heart failure and dysfunction in pulmonary hypertension (PH) in a subject comprising administering a therapeutically effective amount of a vector to the subject, wherein the vector comprises a nucleic acid sequence comprising a cardiomyocyte-specific, cardiac stress-induced promoter operably linked to a MTS and/or a gene that encodes a PH therapeutic, wherein the PH therapeutic is expressed in cardiomyocytes undergoing cardiac stress.

Disclosed are methods of treating dysfunctional cardiomyocytes in a subject comprising administering a therapeutically effective amount of a vector to the subject, wherein the vector comprises a nucleic acid sequence comprising a cardiomyocyte-specific, cardiac stress-induced promoters operably linked to a MTS and a gene that encodes a therapeutic, wherein the therapeutic is expressed in cardiomyocytes undergoing cardiac stress.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a schematic demonstrating fatty acid oxidation and key enzymes in beta-oxidation pathway. HADHA or α-subunit of trifunctional protein carries the 2,3-enoyl-CoA hydratase and the 3-hydroxyacyl-CoA dehydrogenase activities, while HADHB or β-subunit of trifunctional protein bears the 3-ketoacyl-CoA thiolase activity.

FIG. 2 shows an example of RV dysfunction in preclinical PH models. Adult SD rats exposed to 3 wks of hypoxia (10% FiO₂), followed by 4 wks of normoxia (PH). Controls (CON) injected with vehicle and kept in normoxia¹⁻³. Hemodynamics and echocardiography were performed at 7 wks, demonstrating increase RV systolic pressure, impaired RV systolic and diastolic function. Tricuspid annular plane systolic excursion (TAPSE); Cardiac Output (CO); RV late diastolic tissue Doppler velocity (e′), RV end diastolic pressure (RVEDP). n=8-12 rats/group (both male and female). Mean±SEM. * p<0.05.

FIG. 3 shows Decreased Mito FAO respiration in RV muscle fibers of PH rats. Tissue-specific oxygen flux recorded in real time from permeabilized RV of Control (CON) and PH rats (7 wks timepoint). F: FAO (F-junction substrates); F(N): FAO with low concentration of malate (a N-linked substrate); FN: FAO is combined with high concentration of N-linked substrates, i.e., Pyruvate (P) and Glutamate (G); FNS: FN plus succinate (FN- and succinate-linked). ETC: Electron Transfer Capacity. PalM: Palmitoylcartinine/Malate; Oct: Octanoylcarnitine; S: Succinate; Rot: Rotenone. n=6 rats/group, Mean±SEM, * p<0.01.

FIG. 4 shows decreased HADHA and HADHB expression in RV of PH rats (7 wks). RV tissue lysates determining protein level. Western blots and quantitated data. n=6 rats/group. Mean±SEM. * p<0.05.

FIGS. 5A and 5B show reduced mature cardiolipin and less dense cristae packing in RV myocardium of PH rats (7 wks). [FIG. 5A] Mature cardiolipin measurements by mass spectrometry from RV tissue slices. n=4-5/group. Mean±SEM. * p<0.05. [FIG. 5B]. Representative TEM images of RV mito show less dense cristae in RV of PH rats. Scale bar=200 nm.

FIGS. 6A and 6B show reduced complex III (CIII) and IV (CIV) abundance in the supercomplex (SC) region (but not in total expression) in RV myocardium of PH rats (7 wks). FIG. 6A Representative images of blue native page to assay for CIII and CIV SC abundance. FIG. 6B Quantitated data from A. n=10-11 rats/group. Mean±SEM. * p<0.05.

FIG. 7 shows expression of HADHA and HADHB is decreased in hypoxia vs. normoxia. RNA was extracted from H9c2 cells that were cultured in normoxia or hypoxia for 72 hrs and was used for RNA extraction and real-time PCR. n=3 per group. Mean±SEM. * p<0.05. Data were normalized to GAPDH.

FIGS. 8A-8C show HADHA overexpression attenuates the reduced FAO and mature CL amount in hypoxia. [FIG. 8A] HADHA expression. H9c2 cells were infected with adenovirus expressing HADHA (Ad-HADHA) or EGFP (Ad-EGFP) and kept in normoxia or hypoxia for 72 hrs, followed by RNA extraction and real-time PCR. Data were normalized to GAPDH. n=2 each. [FIG. 8B] Effect of HADHA on FAO. Cells from A were used for FAO-linked respiration assays by Oroboros. n=4 each. [FIG. 8C] Effect of HADHA on CL. Cells from A were used for mature CL measurements. n=6. Mean±SEM. * p<0.05.

FIGS. 9A and 9B show knockdown of HADHA is sufficient to decrease levels of mature CL. HADHA was knocked down using sequence-specific shRNA in H9c2 cells. FIG. 9A HADHA expression level in cells with scramble or HADHA shRNA. The vertical line indicates bands from same blot with removing other conditions in between. FIG. 9B Mature CL amount in cells with HADHA knockdown in comparison to control cells. n=3 each. Mean±SEM. * p<0.05.

FIGS. 10A-10F show AAV9 delivered, PH-induced, RV cardiomyocyte-specific, mitochondria-targeted payload delivery in vivo. AAV9-hBNP-MTS-EGFP (13×10¹³ genome copies/kg) was injected in rats 2 days before PH induction and tissues were collected at 4 wks.

FIG. 10A Tiled image of whole heart in cross section. FIG. 10B, FIG. 10C A enlarged area of RV and LV, respectively. FIG. 10D EGFP expression in LV and RV of Ctr and PH rats injected with same amount of AAV9. FIG. 10E EGFP expression in whole cell lysates (WCL) and mitochondrial and cytosolic fractionations from RV of the PH rat injected with AAV9. [FIG. 10F] EGFP expression in major organs of the PH rat.

FIG. 11 shows human BNP promoter (hBNP) can be activated in vitro by phenylephrine (PE). H9c2 cells were transfected with pGL3 basic EGFP (without promoter) or pGL3 with hBNP-driven EGFP and then stimulated with or without PE.

FIG. 12 shows a schematic of trifunctional protein (HADHA and HADHB) expression leads to right ventricle (RV) dysfunction.

FIG. 13 shows an example of viruses that can be used and in vitro study designs.

FIG. 14 shows an example of an efficiency study in vivo.

FIGS. 15A and 15B show (FIG. 15A) schematics of an example of disclosed compositions. (FIG. 15B) Illustration of gene construct that is packaged into AAV9 for specific gene delivery to mitochondria of diseased RV cardiomyocytes in PH.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a gene of interest” includes a plurality of such genes of interest, reference to “the nucleic acid sequence” is a reference to one or more nucleic acid sequences and equivalents thereof known to those skilled in the art, and so forth.

The expression “operably linked” means that the promoter sequence is positioned relative to the coding sequence of the gene of interest such that transcription is able to start. This means that the promoter is positioned upstream of the coding sequence, at a distance enabling the expression of the coding sequence.

The term “percent (%) homology” is used interchangeably herein with the term “percent (%) identity” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62matrix. (See, e.g., Altschul, S. F., et al., Nucleic Acids Res.25:3389-3402, 1997.) A preferred alignment of selected sequences in order to determine “% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

The terms “variant” and “mutant” are used interchangeably herein. As used herein, the term “mutant” refers to a modified nucleic acid or protein which displays the same characteristics when compared to a reference nucleic acid or protein sequence. A variant can be at least 65, 70, 75, 80, 85, 90, 95, or 99 percent homologues to a reference sequence. In some aspects, a reference sequence can be a human pro-B-type natriuretic protein (hBNP) promoter nucleic acid sequence or a wild type human pro-B-type natriuretic protein (hBNP) promoter protein sequence. A “variant” can mean a difference in some way from the reference sequence other than just a simple deletion of an N- and/or C-terminal nucleotide. Variants can also or alternatively include at least one substitution and/or at least one addition, there may also be at least one deletion. Alternatively or in addition, variants can comprise modifications, such as non-natural residues at one or more positions with respect to a reference nucleic acid or protein.

Substitutions, deletions, insertions or any combination thereof may be used to arrive at a final derivative or variant. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3, 4 nucleotide base changes as compared to the parent or reference sequence of the invention, and shares or improves biological function, specificity and/or activity of the parent sequence. Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence (e.g. wild-type sequence, a human pro-B-type natriuretic protein (hBNP) promoter nucleic acid sequence or a human pro-B-type natriuretic protein (hBNP) promoter protein sequence).

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

By an “effective amount” of a composition as provided herein is meant a sufficient amount of the composition to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of ordinary skill in the art using only routine experimentation.

By “treat” is meant to administer a peptide, nucleic acid, vector, or composition of the invention to a subject, such as a human or other mammal (for example, an animal model), that has an increased susceptibility for developing heart dysfunction and failure induced by cardiac stress such as pulmonary hypertension, or that has heart dysfunction and failure induced by cardiac stress such as pulmonary hypertension, in order to prevent or delay a worsening of the effects of the disease or condition, or to partially or fully reverse the effects of the disease.

By “prevent” is meant to minimize the chance that a subject who has an increased susceptibility for developing heart dysfunction and failure induced by cardiac stress such as pulmonary hypertension.

As used herein, the term “subject” or “patient” can be used interchangeably and refer to any organism to which a nucleic acid, vector or composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

As used herein, the terms “administering” and “administration” refer to any method of providing a disclosed composition, nucleic acid, or vector to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed protein so as to treat a subject or induce an immune response. In an aspect, the skilled person can also alter or modify an aspect of an administering step so as to improve efficacy of a disclosed nucleic acid, vector, composition, or a pharmaceutical preparation.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Nucleic Acid Sequences

Disclosed are nucleic acid sequences comprising a cardiomyocyte-specific, cardiac stress-induced promoter operably linked to a mitochondrial targeting sequence (MTS). Disclosed are nucleic acid sequences comprising a cardiomyocyte-specific, cardiac stress-induced promoter operably linked to a gene of interest. Disclosed are nucleic acid sequences comprising a cardiomyocyte-specific, cardiac stress-induced promoter operably linked to a mitochondrial targeting sequence (MTS) and a gene of interest. In some aspects, the entire nucleic acid sequence can be referred to as a nucleic acid construct and each region within the nucleic acid construct can be referred to as a nucleic acid sequence. For example, a nucleic acid construct can comprise a nucleic acid sequence comprising a cardiomyocyte-specific, cardiac stress-induced promoter operably linked to a nucleic acid sequence that encodes a mitochondrial targeting sequence (MTS) and a nucleic acid sequence that encodes a gene of interest.

Disclosed are nucleic acid sequences comprising a human pro-B-type natriuretic protein (hBNP) promoter operably linked to a MTS, a gene of interest, or both.

### 1. Cardiomyocyte-Specific, Cardiac Stress-Induced Promoter

Disclosed are cardiomyocyte-specific, cardiac stress-induced promoters. In some aspects, these promoters are unique from just cardiomyocyte-specific promoters, such as cardiac troponin-T promoter or alpha-myosin heavy chain promoter, because those cardiomyocyte-specific promoters are not cardiac stress-induced (or cardiac disease-induced). Examples of cardiomyocyte-specific, cardiac stress-induced promoters can be, but are not limited to, B-type natriuretic peptide (BNP) promoter (also known as brain-type natriuretic peptide promoter) and atrial natriuretic peptide (ANP) promoter.

In some aspects, the cardiomyocyte-specific, cardiac stress-induced promoters are human specific. Thus, disclosed is a human pro-B-type natriuretic protein (hBNP) promoter. In some aspects as described herein, the hBNP promoter comprises nucleotides 14 to 1931 from accession number U34833. In some aspects as described herein, the human ANP promoter comprises nucleotides −2593 to +100 of the ANP gene (Gene ID: 4878), in which the nucleotide position corresponds to the transcription start site (TSS) that is designated as position +1. In some aspects, a BNP promoter from any species, including but not limited to, mouse, rat, rabbit, pig, and primate, can be used.

In some aspects, the promoter can be a DNA sequence surrounding the first transcribed exon of a gene isoform that is required for recruitment of RNA Pol II to the genomic DNA for transcription initiation. The nucleotide position on the genomic DNA that corresponds to the start of transcription, the transcription start site (TSS), is designated as position +1, and all other core promoter motif positions are designated relative to the +1 TSS position. Thus, the BNP promoter for most species can be within the DNA sequence range from −2,000 to +200 of the BNP gene of the corresponding species, Mouse Gene ID: 18158; Rat Gene ID: 25105; Rabbit Gene ID: 103352511; Pig Gene ID: 396844; Primate Gene ID: 101134730.

In some aspects, the ANP promoter for most species can be within the DNA sequence range from −2,600 to +200 of the BNP gene of the corresponding species. For human ANP promoter, it would be −2593 to +100 of human ANP Gene ID: 4878. ANP gene IDs for other species are: Mouse ID: 230899; Rat ID: 24602; Rabbit ID: 100009087; Pig ID: 397496; Primate (Rhesus Monkey) ID: 714994.

In some aspects, a BNP is produced and expressed in cardiomyocytes. Importantly, ventricular expression of the BNP is downregulated after birth and is strongly increased in adult ventricular cardiomyocytes under cardiac stress such as in PH. Therefore, a gene of interest that is driven by a BNP promoter can lead to cardiomyocyte-specific expression and, importantly, has a low expression in non-stressed cardiomyocytes, but has strong expression in stressed cardiomyocytes.

In some aspects, disclosed is a hBNP promoter comprising the 5′ flanking region (−1818 to +100) of DNA that is adjacent to the 5′ end of the hBNP gene and contains the promoter of the gene. In some aspects, the hBNP promoter is 1918 bps. In some aspects, hBNP promoter can be base pair 14 to 1931 of accession number U34833. Thus, in some aspects, the hBNP promoter comprises the sequence of

(SEQ ID NO: 1)
GTAGAAACACCTTGTGATCACCCTGGCAGTGATTATGAGCTTCAGGTCTGGAATCA
GACTGCTGGCTAGACTAATCAGACTGGTTAGAATCCAGGATTTATCATGTGTCAATT
GTGTGACTTTTGGAAAGTAGATTAATTCATGAACACCATTTCCTCCTCTGAAGTGAG
GAATAATAACCGTGCTTTTCTCACCTCAGGGGCAGATGCTATTTTTTAGGCAAGATC
TGCTTAGAGGTCCCAGTTTCTTATTGCTGCCCTTCTCTGCTGTAACTCTTCTCCCCTC
ATAGACAGCTCCACTCCTCCAGCCTGCTGCTTGTTGACACCAATTCTCTGGAAGGGG
AGTGACATCAGTCATATATGCTTTAGGGGGGTATTTAAGCTGCTATGACTCTTCTCA
GGGGCATTTCTCTCCAAAGTCTCACTTCTAATCACCAGGCCACCTGCTAATGATAAT
TAGATCATGGGTGGTCAGATGAAGGAGGCACTGGGAGAGGGGAAATCCCCATATCT
CTGGTATCCCAGGAAATAGATAACCATCATTCCAGCCATCCTTTTGTTTTCTTTCTTT
CTTTCTTTCTTTCTTACTTTCTTTCTTTCTTTCTTTCCTTCTCTCTTTCTTCCTTCCGCTC
TCTCTCTGTCAACCAGGCTGGAGTGCAGTGGCGTGATCTCAGCTCACTGCAACCTCC
ACCTCCTGGGTTCAAGTGATTCTCCTTCCTCAGCCTCCCGAGTAGCTGGGACTACAG
GCGCCTGCCACCATGCCCAGCTAATTTTTGGTAATTTTAGTAGAGACGGGGTTTCAC
CGTGGTCTCGATCTCCTGACCTCGTGATCCGACCGCCTCGGCCTCTCAAAGTGCTGG
GATTACAGGCGTGAACCACCATGCCCAGCCTATCCTTTTGTTTTCCATCCTGTGTTG
GCTTGGTGGGGGAGAGGAGGTGTTGACACGTGGAGGACACACATATAAGGCATTCT
TGGGTGACTTCGTCATCACTGGACCCTATCTCTCAAAATTCCAGCGAAATCTGCTCT
TCCCTTTAAGGAGTGAAAGAAGGGTCAGCATTCCAGAAGTTCCTGGTCATACCCAG
GCTTTTAATGAATTGCCACTGGGGAATCAGCATCCCGTTGCTGTAAGGACTATAAGA
TGGCGGATTGTGAGAGCATAGGGAAAGGTCTCGGAGGTCTCTTGTCCTTGCTCCAC
GCAGGTCTTTCTGGCCTGAAAATCCCGTTGAAGAGAGCAGCTCTTGAGAGTTTGCTC
CAAGTTCCCTCGGGGTGATCAGCACCACGGACAGGGGCCAGGGCGCCCCCGAGGAC
CCGCAGGCAGGCAGGGTGCACAGCGGGGAGCAGGTGCTGCGCTACGTGCGGGCCA
GGGAACTCGCGCGGGGAGGGGAGAGGCGCCGCGGGTGGCGGGGTCTTGGCCGGGG
CTGTTTTCGCTGTGAGATCACCCCGTGCTCCCAGCGCTCACGTCGGTCCTCGGAAAG
CCGGGGTCCTCCCTGCCTTTTCCAGCAACGGTGGGGTGGGGAGGCAGGAAGAAAGC
GCCAACCTAGGACCCCGGAGATTTGCAGCAAAGGAAGAAGCGGGAGACGGGCACT
TGTCTGTGTCTCCAGCGCGTTCCTGCCCCCCGCCGACCCGGCCCATTTCTATACAAG
GTCGCTCTGCCCGGTCTCCACCTCCCACGTGCAGGCCGCGGAGGGGCTCATTCCCGG
GCCCTGATCTCAGAGGCCCGGAATGTGGCTGATAAATCAGAGACTAGACCTGCATG
GCAGGCAGGCCCGACACTCAGCTCCAGGATAAAAGGCCACGGTGTCCCGAGGAGC
CAGGAGGAGCACCCCGCAGGCTGAGGGCAGGTGGGAAGCAAACCCGGACGCATCG
CAGCAGCAGCAGCAGCAGCAGAAGCAGCAGCAGCAGCCTCCGCAGTCCC

In some aspects, altered promoter sequences can be used, wherein the altered promoter sequence retains promoter activity. In some aspect, an altered promoter sequence can be 70, 75, 80, 85, 90, 95, or 99% identical to SEQ ID NO:1. In some aspects, a functional fragment of the disclosed promoters can be used. A functional fragment can be any fragment of SEQ ID NO:1 that retains promoter activity.

In some aspects, a cardiomyocyte-specific, cardiac stress-induced promoter, such as a BNP promoter, in the presence of PH, drives gene expression preferentially in the diseased right ventricle (RV) cardiomyocytes upon PH-induced RV stress but little gene expression in left ventricular cardiomyocytes. Thus, the disclosed cardiomyocyte-specific, cardiac stress-induced promoters have advantages in comparison to the currently widely used cardiac promoters such as cardiac troponin T promoter that continuously drive gene expression in whole ventricles (i.e., both left and right ventricular cardiomyocytes regardless of whether there is cardiac stress or not).

### 2. Mitochondrial Targeting Sequence

In some aspects, the cardiomyocyte-specific, cardiac stress-induced promoter is operably linked to a MTS. In some aspects, the cardiomyocyte-specific, cardiac stress-induced promoter controls or drives expression of a MTS. In some aspects, the disclosed nucleic acid sequences comprise a MTS operably linked to a cardiomyocyte-specific, cardiac stress-induced promoter. A MTS can also be referred to as a mitochondrial localization signal. In some aspects, the MTS can be any known MTS. For example, the MTS can be, but is not limited to, a nucleic acid sequence that encodes those peptides listed in Table 1.

TABLE 1
Examples of MTS sequences

| | |
|---|---|
| hsCOX10 | MAASPHTLSSRLLTGCVGGSVWYLERRT (SEQ ID NO: 3) |
| scRPM2 | MAFKSFIYSKGYHRSAAQKKTATSFFDSSYQYLRQNQGLVNSDPVLHASHLHPHPVV |
| | VANVNYNNVDDILHPHDLDSSINNTNNPLTHEELLYNQNVSLRSLKQQQSTNYVNNN |
| | NNNQHRYY (SEQ ID NO: 4) |
| lcSirt5 | MRKRSLRCHLWSANASLSPRKDEVTSRKESENLVKGKKNKKSHLHLLLFTASKIGTDS |
| | VFDVQKSKECCKELGLLFTSLIHSIGSFPFDEEPKAAAVFLPGSLPQLTVLVLAPGSGSC |
| | PTGKSTPHLAASGRNAELLRPQNSMIVRQFTCRGTISSHLCAHLRKPHDSRNMARP |
| | (SEQ ID NO: 5) |
| tbNDUS7 | MLRRTSFNFTGRAMISRGSPEWSHRLDLKKGKKTTMMHKLGTSKPNNALQYAQMTL |
| | (SEQ ID NO: 6) |
| ncQCR2 | MISRSALSRGSQLALRRPAAAKTAQRGFAAAAASPAASYEPTTIAG (SEQ ID NO: 7) |
| hsATP5G2 | MPELILYVAITLSVAERLVGPGHACAEPSFRSSRCSAPLCLLCSGSSSPATAPHPLKMFA |
| | CSKFVSTPSLVKSTSQLLSRPLSAVVLKRPEILTDESLSSLAVSCPLTSLVSSRSFQTSAIS |
| | RDIDTA (SEQ ID NO: 8) |
| hsLACTB | MYRLMSAVTARAAAPGGLASSCGRRGVHQRAGLPPLGHGWVGGLGLGLGLALGVK |
| | LAGGLRGAAPAQSPAAPDPEASPLAEPPQEQSLAPWSPQTPAPPCSRCFARAIESSRDLL |
| | (SEQ ID NO: 9) |
| spilv1 | MTVLAPLRRLHTRAAFSSYGREIALQKRFLNLNSCSAVRRYGTGFSNNLRIKKLKNAF |
| | GVVRANSTKSTSTVTTASPIKYDSSFVGKTGGEIFHDMMLKHNVKHVFGYPGGAILPV |
| | FDAIYRSPHFEFILPRHEQAAGHA (SEQ ID NO: 10) |
| gmCOX2 | MILCPLEAFIVQHILTISVMGLLSCFRSTVLRKCSKGSSGMSRFLYTNNFQRNLISSGGN |
| | ESYYGYFNRRSYTSLYMGTGTVGGITSARIRVPNVGCEGFMCSSHLSITQRNSRLIHSTS |
| | KIVPN (SEQ ID NO: 11) |
| crATP6 | MALQQAAPRVFGLLGRAPVALGQSGILTGSSGFKNQGFNGSLQSVENHVYAQAFSTSS |
| | QEEQAAPSIQGASGMKLPGMAGSMLLGKSRSGLRTGSMVPFAAQQAMNM (SEQ ID |
| | NO: 12) |
| hsOPA1 | MWRLRRAAVACEVCQSLVKHSSGIKGSLPLQKLHLVSRSIYHSHHPTLKLQRPQLRTS |
| | FQQFSSLTNLPLRKLKFSPIKYGYQPRRN (SEQ ID NO: 13) |
| hsSDHD | MAVLWRLSAVCGALGGRALLLRTPVVRPAHISAFLQDRPIPEWCGVQHIHLSPSHH |
| | (SEQ ID NO: 14) |
| hsADCK3 | MAAILGDTIMVAKGLVKLTQAAVETHLQHLGIGGELIMAARALQSTAVEQIGMFLGK |
| | VQGQDKHEEYFAENFGGPEGEFHFSVPHAAGASTDFSSASAPDQSAPPSLGHAHSEGP |
| | APAYVASGPFREAGFPGQASSPLGRANGRLFANPRDSFSAMGFQRRF (SEQ ID NO: 15) |
| osP0644B06.2 | MALLLRHSPKLRRAHAILGCERGTVVRHFSSSTCSSLVKEDTVSSSNLHPEYAKKIGGS |
| 4-2 | DFSHDRQSGKELQNFKVSPQEASRASNFMRASKYGMPITANGVHSLFSCGQVVPSRCF |
| | (SEQ ID NO: 16) |
| Neurospora | MASTRVLASRLASQMAASAKVARPAVRVAQVSKRTIQTGSPLQTLKRTQMTSIVNAT |
| crassa ATP9 | TRQAFQKRA (SEQ ID NO: 17) |
| (ncATP9) | |
| hsGHITM | MLAARLVCLRTLPSRVFHPAFTKASPVVKNSITKNQWLLTPSRE (SEQ ID NO: 18) |
| hsNDUFAB1 | MASRVLSAYVSRLPAAFAPLPRVRMLAVARPLSTALCSAGTQTRLGTLQPAL VLAQVP |
| | GRVTQLCRQY (SEQ ID NO: 19) |
| hsATP5G3 | MFACAKLACTPSLIRAGSRVAYRPISASVLSRPEASRTGEGSTVENGAQNGVSQLIQRE |
| | FQTSAISR (SEQ ID NO: 20) |
| crATP6_ | MALQQAAPRVFGLLGRAPVALGQSGILTGSSGFKNQGFNGSLQSVENHVYAQAFSTSS |
| hsADCK3 | QEEQAAPSIQGASGMKLPGMAGSMLLGKSRSGLRTGSMVPFAAQQAMNMGGMAAIL |
| | GDTIMVAKGLVKLTQAAVETHLQHLGIGGELIMAARALQSTAVEQIGMFLGKVQGQD |
| | KHEEYFAENFGGPEGEFHFSVPHAAGASTDFSSASAPDQSAPPSLGHAHSEGPAPAYV |
| | ASGPFREAGFPGQASSPLGRANGRLFANPRDSFSAMGFQRRFGG (SEQ ID NO: 21) |
| ncATP9_ncATP9 | MASTRVLASRLASQMAASAKVARPAVRVAQVSKRTIQTGSPLQTLKRTQMTSIVNAT |
| | TRQAFQKRAMASTRVLASRLASQMAASAKVARPAVRVAQVSKRTIQTGSPLQTLKRT |
| | QMTSIVNATTRQAFQKRA (SEQ ID NO: 22) |
| zmLOC100282174 | MALLRAAVSELRRRGRGALTPLPALSSLLSSLSPRSPASTRPEPNNPHADRRHVIALRR |
| | CPPLPASAVLAPELLHARGLLPRHWSHASPLSTSSSSSRPADKAQLTWVDKWIPEAARP |
| | Y (SEQ ID NO: 23) |
| ncATP9_ | MASTRVLASRLASQMAASAKVARPAVRVAQVSKRTIQTGSPLQTLKRTQMTSIVNAT |
| zmLOC100282174_ | TRQAFQKRAMALLRAAVSELRRRGRGALTPLPALSSLLSSLSPRSPASTRPEPNNPHAD |
| spilv1_ncATP9 | RRHVIALRRCPPLPASAVLAPELLHARGLLPRHWSHASPLSTSSSSSRPADKAQLTWVD |
| | KWIPEAARPYMTVLAPLRRLHTRAAFSSYGREIALQKRFLNLNSCSAVRRYGTGFSNN |
| | LRIKKLKNAFGVVRANSTKSTSTVTTASPIKYDSSFVGKTGGEIFHDMMLKHNVKHVF |
| | GYPGGAILPVFDAIYRSPHFEFILPRHEQAAGHAMASTRVLASRLASQMAASAKVARP |
| | AVRVAQVSKRTIQTGSPLQTLKRTQMTSIVNATTRQAFQKRA (SEQ ID NO: 24) |
| zmLOC100282174_ | MALLRAAVSELRRRGRGALTPLPALSSLLSSLSPRSPASTRPEPNNPHADRRHVIALRR |
| hsADCK3_ | CPPLPASAVLAPELLHARGLLPRHWSHASPLSTSSSSSRPADKAQLTWVDKWIPEAARP |
| crATP6_ | YMAAILGDTIMVAKGLVKLTQAAVETHLQHLGIGGELIMAARALQSTAVEQIGMFLG |
| hsATP5G3 | KVQGQDKHEEYFAENFGGPEGEFHFSVPHAAGASTDFSSASAPDQSAPPSLGHAHSEG |
| | PAPAYVASGPFREAGFPGQASSPLGRANGRLFANPRDSFSAMGFQRRFMALQQAAPR |
| | VFGLLGRAPVALGQSGILTGSSGFKNQGFNGSLQSVENHVYAQAFSTSSQEEQAAPSIQ |
| | GASGMKLPGMAGSMLLGKSRSGLRTGSMVPFAAQQAMNMMFACAKLACTPSLIRAG |
| | SRVAYRPISASVLSRPEASRTGEGSTVENGAQNGVSQLIQREFQTSAISR (SEQ ID |
| | NO: 25) |
| zmLOC100282174_ | MALLRAAVSELRRRGRGALTPLPALSSLLSSLSPRSPASTRPEPNNPHADRRHVIALRR |
| hsADCK3_ | CPPLPASAVLAPELLHARGLLPRHWSHASPLSTSSSSSRPADKAQLTWVDK WIPEAARP |
| hsATP5G3 | YMAAILGDTIMVAKGLVKLTQAAVETHLQHLGIGGELIMAARALQSTAVEQIGMFLG |
| | KVQGQDKHEEYFAENFGGPEGEFHFSVPHAAGASTDFSSASAPDQSAPPSLGHAHSEG |
| | PAPAYVASGPFREAGFPGQASSPLGRANGRLFANPRDSFSAMGFQRRFMFACAKLACT |
| | PSLIRAGSRVAYRPISASVLSRPEASRTGEGSTVFNGAQNGVSQLIQREFQTSAISR (SEQ |
| | ID NO: 26) |
| ncATP9_ | MASTRVLASRLASQMAASAKVARPAVRVAQVSKRTIQTGSPLQTLKRTQMTSIVNAT |
| zmLOC100282174 | TRQAFQKRAMALLRAAVSELRRRGRGALTPLPALSSLLSSLSPRSPASTRPEPNNPHAD |
| | RRHVIALRRCPPLPASAVLAPELLHARGLLPRHWSHASPLSTSSSSSRPADKAQLTWVD |
| | KWIPEAARPY (SEQ ID NO: 27) |
| hsADCK3_ | MAAILGDTIMVAKGLVKLTQAAVETHLQHLGIGGELIMAARALQSTAVEQIGMFLGK |
| zmLOC100282174_ | VQGQDKHEEYFAENFGGPEGEFHFSVPHAAGASTDFSSASAPDQSAPPSLGHAHSEGP |
| crATP6_ | APAYVASGPFREAGFPGQASSPLGRANGRLFANPRDSFSAMGFQRRFMALLRAAVSEL |
| hsATP5G3 | RRRGRGALTPLPALSSLLSSLSPRSPASTRPEPNNPHADRRHVIALRRCPPLPASAVLAP |
| | ELLHARGLLPRHWSHASPLSTSSSSSRPADKAQLTWVDKWIPEAARPYMALQQAAPR |
| | VFGLLGRAPVALGQSGILTGSSGFKNQGFNGSLQSVENHVYAQAFSTSSQEEQAAPSIQ |
| | GASGMKLPGMAGSMLLGKSRSGLRTGSMVPFAAQQAMNMMFACAKLACTPSLIRAG |
| | SRVAYRPISASVLSRPEASRTGEGSTVENGAQNGVSQLIQREFQTSAISR (SEQ ID |
| | NO: 28) |
| crATP6_ | MALQQAAPRVFGLLGRAPVALGQSGILTGSSGFKNQGFNGSLQSVENHVYAQAFSTSS |
| hsADCK3_ | QEEQAAPSIQGASGMKLPGMAGSMLLGKSRSGLRTGSMVPFAAQQAMNMMAAILGD |
| zmLOC100282174_ | TIMVAKGLVKLTQAAVETHLQHLGIGGELIMAARALQSTAVEQIGMFLGKVQGQDKH |
| hsATP5G3 | EEYFAENFGGPEGEFHFSVPHAAGASTDFSSASAPDQSAPPSLGHAHSEGPAPAYVASG |
| | PFREAGFPGQASSPLGRANGRLFANPRDSFSAMGFQRRFMALLRAAVSELRRRGRGAL |
| | TPLPALSSLLSSLSPRSPASTRPEPNNPHADRRHVIALRRCPPLPASAVLAPELLHARGLL |
| | PRHWSHASPLSTSSSSSRPADKAQLTWVDKWIPEAARPYMFACAKLACTPSLIRAGSR |
| | VAYRPISASVLSRPEASRTGEGSTVENGAQNGVSQLIQREFQTSAISR (SEQ ID NO: 29) |
| hsADCK3_ | MAAILGDTIMVAKGLVKLTQAAVETHLQHLGIGGELIMAARALQSTAVEQIGMFLGK |
| zmLOC100282174 | VQGQDKHEEYFAENFGGPEGEFHFSVPHAAGASTDFSSASAPDQSAPPSLGHAHSEGP |
| | APAYVASGPFREAGFPGQASSPLGRANGRLFANPRDSFSAMGFQRRFGGMALLRAAV |
| | SELRRRGRGALTPLPALSSLLSSLSPRSPASTRPEPNNPHADRRHVIALRRCPPLPASAV |
| | LAPELLHARGLLPRHWSHASPLSTSSSSSRPADKAQLTWVDKWIPEAARPYGG (SEQ |
| | ID NO: 30) |
| hsADCK3_ | MAAILGDTIMVAKGLVKLTQAAVETHLQHLGIGGELIMAARALQSTAVEQIGMFLGK |
| zmLOC100282174_ | VQGQDKHEEYFAENFGGPEGEFHFSVPHAAGASTDFSSASAPDQSAPPSLGHAHSEGP |
| crATP6 | APAYVASGPFREAGFPGQASSPLGRANGRLFANPRDSFSAMGFQRRFGGMALLRAAV |
| | SELRRRGRGALTPLPALSSLLSSLSPRSPASTRPEPNNPHADRRHVIALRRCPPLPASAV |
| | LAPELLHARGLLPRHWSHASPLSTSSSSSRPADKAQLTWVDKWIPEAARPYGGMALQ |
| | QAAPRVFGLLGRAPVALGQSGILTGSSGFKNQGFNGSLQSVENHVYAQAFSTSSQEEQ |
| | AAPSIQGASGMKLPGMAGSMLLGKSRSGLRTGSMVPFAAQQAMNMGG (SEQ ID |
| | NO: 31) |
| ncATP9_ | MASTRVLASRLASQMAASAKVARPAVRVAQVSKRTIQTGSPLQTLKRTQMTSIVNAT |
| zmLOC100282174_ | TRQAFQKRAMALLRAAVSELRRRGRGALTPLPALSSLLSSLSPRSPASTRPEPNNPHAD |
| spilv1_ | RRHVIALRRCPPLPASAVLAPELLHARGLLPRHWSHASPLSTSSSSSRPADKAQLTWVD |
| GNFPncATP9 | KWIPEAARPYMTVLAPLRRLHTRAAFSSYGREIALQKRFLNLNSCSAVRRYGTGFSNN |
| | LRIKKLKNAFGVVRANSTKSTSTVTTASPIKYDSSFVGKTGGEIFHDMMLKHNVKHVF |
| | GYPGGAILPVFDAIYRSPHFEFILPRHEQAAGHAVSGEGDATYGKLTLKFICTTGKLPVP |
| | WPTLVTTLTYGVQCFSRYPDHMKQHDFFKSAMPEGYVQERTIFFKDDGNYKTRAEVK |
| | FEGDTLVNRIELKGIDFKEDGNILGHKLEYNYNSHNVYIMADKQKNGIKVNFKIRHNIE |
| | DGSVQLADHYQQNTPIGDGPVLLPDNHYLSTQSALSKDPNEMASTRVLASRLASQMA |
| | ASAKVARPAVRVAQVSKRTIQTGSPLQTLKRTQMTSIVNATTRQAFQKRA (SEQ ID |
| | NO: 32) |
| ncATP9_ | MASTRVLASRLASQMAASAKVARPAVRVAQVSKRTIQTGSPLQTLKRTQMTSIVNAT |
| zmLOC100282174_ | TRQAFQKRAMALLRAAVSELRRRGRGALTPLPALSSLLSSLSPRSPASTRPEPNNPHAD |
| spilv1_lcSirt5_ | RRHVIALRRCPPLPASAVLAPELLHARGLLPRHWSHASPLSTSSSSSRPADKAQLTWVD |
| osP0644B06.24- | KWIPEAARPYMTVLAPLRRLHTRAAFSSYGREIALQKRFLNLNSCSAVRRYGTGFSNN |
| 2_hsATP5G2_ | LRIKKLKNAFGVVRANSTKSTSTVTTASPIKYDSSFVGKTGGEIFHDMMLKHNVKHVF |
| ncATP9 | GYPGGAILPVFDAIYRSPHFEFILPRHEQAAGHAMRKRSLRCHLWSANASLSPRKDEVT |
| | SRKESENLVKGKKNKKSHLHLLLFTASKIGTDSVFDVQKSKECCKELGLLFTSLIHSIGS |
| | FPFDEEPKAAAVFLPGSLPQLTVLVLAPGSGSCPTGKSTPHLAASGRNAELLRPQNSMI |
| | VRQFTCRGTISSHLCAHLRKPHDSRNMARPMALLLRHSPKLRRAHAILGCERGTVVRH |
| | FSSSTCSSLVKEDTVSSSNLHPEYAKKIGGSDFSHDRQSGKELQNFKVSPQEASRASNF |
| | MRASKYGMPITANGVHSLFSCGQVVPSRCFMPELILYVAITLSVAERLVGPGHACAEP |
| | SFRSSRCSAPLCLLCSGSSSPATAPHPLKMFACSKFVSTPSLVKSTSQLLSRPLSAVVLK |
| | RPEILTDESLSSLAVSCPLTSLVSSRSFQTSAISRDIDTAMASTRVLASRLASQMAASAK |
| | VARPAVRVAQVSKRTIQTGSPLQTLKRTQMTSIVNATTRQAFQKRA (SEQ ID NO: 33) |

In some aspects, the MTS encodes residues 1-40 of the amino acid sequence of very long chain acyl-CoA dehydrogenase. For example, a MTS nucleic acid sequence can comprises or consist of the nucleic acid sequence:

(SEQ ID NO: 2)
ATGCAGGCGGCTCGGATGGCCGCGAGCTTGGGGCGGCAGCTGCTGAGGC
TCGGGGGCGGAAGCTCGCGGCTCACGGCGCTCCTGGGGCAGCCCCGGCC
CGGCCCTGCCCGGCGGCCCTATGCCGGG.

In some aspects, the MTS operably linked to a cardiomyocyte-specific, cardiac stress-induced promoter is also linked to a gene of interest. In some aspects, the MTS is present at the 5′ end of a gene of interest. In some aspects, there can be a cleavage site present to cleave the gene of interest once inside the mitochondria. In some aspects, the cleavage site can be present between the MTS and the gene of interest.

### 3. Gene of Interest

In some aspects, the cardiomyocyte-specific, cardiac stress-induced promoter controls or drives expression of a MTS and a gene of interest. In some aspects, the disclosed nucleic acid sequences disclosed herein further comprise a gene of interest. In some aspects, the gene of interest is a gene that encodes a therapeutic. In some aspects, the gene of interest is a gene that encodes a pulmonary hypertension (PH) therapeutic. In some aspects, the PH therapeutic can be, but are not limited to, hydroxyacyl-CoA dehydrogenase trifunctional multienzyme complex subunit alpha (HADHA) or Hydroxyacyl-CoA Dehydrogenase Trifunctional Multienzyme Complex Subunit Beta (HADHB). Example sequences of HADHA, can be, but are not limited to, (Human) NM_000182.5, (Rat) NM_130826.2, and (Mouse) NM_178878.3. Example sequences of HADHB, can be, but are not limited to, (Human) NM_000183.3, (Rat) NM_133618.3, (Mouse) NM 001289798.1, (Mouse) NM_001289799.1, and (Mouse) NM_145558.2

In some aspects, the therapeutic can be c-Src. Examples of c-Src can be, but are not limited to, (Human) NM_005417.5, (Human) NM_198291.3, (Rat) NM_031977.1, (Mouse) NM_001025395.2, and (Mouse) NM_009271.3. Mitochondrial c-Src can improve energy function in the heart in diseases such as PH.

### C. Vectors

Disclosed are vectors comprising any of the nucleic acid sequences disclosed herein.

Disclosed herein is a tool for specific gene manipulation in mitochondria of diseased RV cardiomyocytes in the heart in PH by combining three elements: vector; cardiomyocyte-specific, cardiac stress-induced promoter; and mitochondrial targeted sequences (MTS) (i.e., Adeno-associated virus (AAV) 9, human pro-B-type natriuretic protein (hBNP) promoter, and MTS). In some aspects, the vector comprises the cardiomyocyte-specific, cardiac stress-induced promoter and the MTS.

In some aspects, the vector comprises the cardiomyocyte-specific, cardiac stress-induced promoter and a gene of interest.

The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, the vector can be a viral vector. For example, the viral vector can be an adeno-associated viral vector (AAV). In some aspects, the AAV can be AAV9. In some aspects, the vector can be a non-viral vector, such as a DNA based vector.

In some aspects, the vector is AAV9. In some aspects, a benefit of using AAV vectors can be that AAV is non-pathogenic in human and elicits a very mild immune response. In some aspects, the disclosed vectors are considered recombinant vectors. A recombinant AAV can lack two essential genes for viral integration and replication but remains primarily episomal and can persist in non-dividing cells for long periods of time. The tissue specificity of AAV can be determined by the viral capsid serotype, which allows targeting the gene of interest to specific tissues. In some aspects, AAV9 can be used to efficiently target the heart, although it also can target to other organs/tissues such as muscles, lungs, kidneys. In some aspects, to overcome the non-heart specific targeted gene expression by AAV9, gene expression can be driven by a cardiomyocyte-specific, cardiac stress-induced promoter, such as hBNP promoter.

### 1. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence comprising a cardiomyocyte-specific, cardiac stress-induced promoter operably linked to a nucleic acid sequence encoding a MTS and a PH therapeutic.

The “control elements” present in an expression vector are those non-translated regions of the vector—enhancers, promoters (e.g., a human pro-B-type natriuretic protein (hBNP) promoter), 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes ß-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into its nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically, a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### D. Compositions

Disclosed are compositions comprising the nucleic acid sequences, vectors or cells disclosed herein. Disclosed are compositions comprising a nucleic acid construct, wherein the nucleic acid construct comprises a nucleic acid sequence encoding a cardiomyocyte-specific, cardiac stress-induced promoter operably linked to a nucleic acid sequence encoding a MTS and/or a therapeutic (i.e. a gene of interest). Disclosed are compositions comprising a nucleic acid construct, wherein the nucleic acid sequence comprises a nucleic acid sequence encoding a cardiomyocyte-specific, cardiac stress-induced promoter operably linked to a nucleic acid sequence encoding a MTS and/or a PH therapeutic. Also disclosed are compositions comprising a vector, such as a viral vector, comprising a nucleic acid construct, wherein the nucleic acid construct comprises a nucleic acid sequence encoding a cardiomyocyte-specific, cardiac stress-induced promoter operably linked to a nucleic acid sequence encoding a MTS and/or a PH therapeutic.

The disclosed compositions can further comprise a pharmaceutically acceptable carrier.

### 1. Delivery of Compositions

In the methods described herein, delivery (or administration) of the nucleic acid sequences or compositions disclosed herein to cells can be via a variety of mechanisms. As defined above, disclosed herein are compositions comprising any one or more of the peptides, nucleic acids, vectors and/or cells described herein can be used to produce a composition which can also include a carrier such as a pharmaceutically acceptable carrier. For example, disclosed are pharmaceutical compositions, comprising the peptides disclosed herein, and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl choline (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, P A 1995. Typically, an appropriate amount of pharmaceutically acceptable salt is used in the formulation to render the formulation isotonic. Other examples of the pharmaceutically acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed delivery techniques can be used not only for the disclosed compositions but also the disclosed nucleic acid sequences and vectors.

### E. Recombinant Cells

Disclosed are recombinant cells comprising one or more of the disclosed nucleic acid constructs or vectors. For example, disclosed are recombinant cells comprising a nucleic acid construct, wherein the nucleic acid construct comprises a nucleic acid sequence encoding a cardiomyocyte-specific, cardiac stress-induced promoter operably linked to a MTS and/or a gene of interest.

In some aspects, the cell is a mammalian cell. In some aspects, the cell is a cardiomyocyte.

### F. Methods

Disclosed are methods of treating a subject in need thereof with one or more of the disclosed nucleic acid sequences or vectors.

In some aspects, the disclosed methods are based on the presence of cardiac stress. Cardiac stress triggers expression from a cardiomyocyte-specific, cardiac stress-induced promoter, such as hBNP. In some aspects, cardiac stress can be any stimulus that can increase myocardial wall stress (e.g., pressure overload, volume overload) and/or any pathophysiological processes such as myocardial ischemia and endocrine (paracrine) modulation by other neurohormones and cytokines. For example, in PH, the cardiac stress includes right ventricular pressure overload and increased cytokines such as endothelin-1. In some aspects, cardiac stress can be assessed by the presence of biomarkers that are elevated such as, but not limited to, atrial natriuretic peptide (ANP), endothelin-1, and cardiac troponins (e.g., troponin T). Other measurable factors can be parameters from echocardiography (e.g., calculated heart weights, ventricular chamber size and wall thickness, cardiac strain, cardiac systolic and diastolic function, etc.).

In some aspects, a sample from a subject can be used to detect the presence of a cardiac stress biomarker prior to administering one of the disclosed vectors to the subject. In some aspects, a sample from a subject can be obtained after administering one of the disclosed vectors to the subject to determine if the therapeutic is effective.

In some aspects, there is a decrease in trifunctional protein (e.g.; HADHA and HADHB) expression in RV myocardium that is associated with RV dysfunction, abnormal RV fatty acid oxidation (FAO), decreased cardiolipin content, and abnormal mitochondrial cristae. Therefore, increasing the levels of certain proteins, such as a trifunctional protein, can be therapeutic to RV dysfunction in response from cardiac stress.

### 1. Treating RV Dysfunction and Failure in Pulmonary Hypertension

Disclosed are methods of treating RV dysfunction and failure in pulmonary hypertension (PH) in a subject comprising administering a therapeutically effective amount of a vector to the subject, wherein the vector comprises a nucleic acid sequence comprising a cardiomyocyte-specific, cardiac stress-induced promoter operably linked to a mitochondrial targeting sequence (MTS) and/or a gene that encodes a PH therapeutic, wherein the PH therapeutic is expressed in cardiomyocytes undergoing cardiac stress. In some aspects, the cardiomyocyte-specific, cardiac stress-induced promoter can be hBNP.

Disclosed are methods of treating RV dysfunction and failure in PH in a subject comprising administering a therapeutically effective amount of a vector to the subject, wherein the vector comprises a nucleic acid sequence comprising a hBNP promoter operably linked to a MTS and/or a gene that encodes a PH therapeutic, wherein the PH therapeutic is expressed in cardiomyocytes undergoing cardiac stress.

In some aspects, the hBNP promoter becomes active under cardiac stress. BNP is produced and expressed in cardiomyocytes. Importantly, ventricular expression of the BNP is downregulated after birth and is strongly increased in adult ventricular cardiomyocytes under cardiac stress such as in PH. Therefore, a gene of interest that is driven by BNP promoter can lead to cardiomyocyte-specific expression and, importantly, has a low expression in non-stressed cardiomyocytes, but a strong expression in stressed cardiomyocytes. PH causes cardiac stress and therefore PH can activate expression from a BNP promoter.

In some aspects, a hBNP promoter as disclosed herein, can be used to treat RV dysfunction and failure in PH because it can drive gene expression preferentially in diseased RV cardiomyocytes upon PH-induced RV stress but little gene expression in left ventricular cardiomyocytes. This approach has many advantages in comparison to the currently widely used cardiac promoters such as cardiac troponin T promoter that continuously drives gene expression in whole ventricles (i.e., both left and right ventricular cardiomyocytes regardless of whether there is cardiac stress or not).

In some aspects, disclosed is a hBNP promoter comprising the 5′ flanking region −1818 to +100 of DNA that is adjacent to the 5′ end of the hBNP gene and contains the promoter of the gene. In some aspects, the hBNP promoter is 1918 bps. In some aspects, hBNP promoter can be base pair 14 to 1931 of accession number U34833. Thus, in some aspects, the hBNP promoter comprises the sequence of SEQ ID NO:1.

In some aspects, altered promoter sequences can be used, wherein the altered promoter sequence retains promoter activity. In some aspect, an altered promoter sequence can be 70, 75, 80, 85, 90, 95, or 99% identical to SEQ ID NO:1. In some aspects, a functional fragment of the disclosed promoters can be used. A functional fragment can be any fragment of SEQ ID NO:1 that retains promoter activity.

In some aspects, the MTS encodes residues 1-40 of the amino acid sequence of very long chain acyl-CoA dehydrogenase. Thus, the MTS nucleic acid sequence can be ATGCAGGCGGCTCGGATGGCCGCGAGCTTGGGGCGGCAGCTGCTGAGGCTCGGGGG CGGAAGCTCGCGGCTCACGGCGCTCCTGGGGCAGCCCCGGCCCGGCCCTGCCCGGC GGCCCTATGCCGGG (SEQ ID NO:2). In some aspects, the MTS can be any MTS described in Table 1.

In some aspects, the MTS operably linked to a cardiomyocyte-specific, cardiac stress-induced promoter is also linked to a gene of interest. In some aspects, the MTS is present at the 5′ end of a gene of interest.

In some aspects, the PH therapeutic is expressed in the right ventricle. In some aspects, the PH therapeutic is not expressed or is expressed significantly less in the left ventricle. In some aspects, the PH therapeutic is right ventricle specific meaning it is only expressed in the right ventricle or at least expressed at a much higher level in the right ventricle compared to the left ventricle. In some aspects, the hBNP promoter drives gene expression predominately in dysfunctional cardiomyocytes. In PH, where dysfunctional cardiomyocytes are present mostly in the right ventricle, gene expression is highly expressed in the right ventricle in comparison to left ventricle when under control of the hBNP promoter. In some aspects, there can be a very low expression level in normal cardiomyocytes in the left ventricle if the hBNP promoters has a low basal activity at baseline. In some aspects, there is expression in other major organs in the body besides the heart, such as, a low expression in liver which is not surprising since hBNP promoter has some activity in liver.

In some aspects, the PH therapeutic can be, but are not limited to, hydroxyacyl-CoA dehydrogenase trifunctional multienzyme complex subunit alpha (HADHA) or Hydroxyacyl-CoA Dehydrogenase Trifunctional Multienzyme Complex Subunit Beta (HADHB). Example sequences of HADHA, can be, but are not limited to, (Human) NM_000182.5, (Rat) NM_130826.2, and (Mouse) NM_178878.3. Example sequences of HADHB, can be, but are not limited to, (Human) NM_000183.3, (Rat) NM_133618.3, (Mouse) NM_001289798.1, (Mouse) NM_001289799.1, and (Mouse) NM_145558.2

In some aspects, the therapeutic can be c-Src. Examples of c-Src can be, but are not limited to, (Human) NM_005417.5, (Human) NM_198291.3, (Rat) NM_031977.1, (Mouse) NM_001025395.2, and (Mouse) NM_009271.3.

In some aspects, the MTS is cleaved from the PH therapeutic after entering a mitochondria. In some aspects, there is a cleavage site between the PH therapeutic and the MTS.

In some aspects, there is little to no expression of the PH therapeutic in healthy cardiomyocytes. In some aspects, the hBNP promoter drives gene expression predominately in dysfunctional cardiomyocytes which are present mostly in the right ventricle during PH. In some aspects, there can be a very low expression level in normal cardiomyocytes in the left ventricle if the hBNP promoter has a low basal activity at baseline. In some aspects, there is expression in other major organs in the body besides the heart, such as a low expression in liver which is not surprising since hBNP promoter has some activity in liver.

In some aspects, the vector is administered via intravenous administration. In some aspects, the administration is via local injection, intracoronary, subcutaneous, intraperitoneal, or inhalation.

### 2. Treating Dysfunctional Cardiomyocytes

Disclosed are methods of treating dysfunctional cardiomyocytes in a subject comprising administering a therapeutically effective amount of a vector to the subject, wherein the vector comprises a nucleic acid sequence comprising a cardiomyocyte-specific, cardiac stress-induced promoters operably linked to a mitochondrial targeting sequence (MTS) and/or a gene that encodes a therapeutic, wherein the therapeutic is expressed in cardiomyocytes undergoing cardiac stress. In some aspects, the cardiomyocyte-specific, cardiac stress-induced promoter can be hBNP.

Disclosed are methods of treating dysfunctional cardiomyocytes in a subject comprising administering a therapeutically effective amount of a vector to the subject, wherein the vector comprises a nucleic acid sequence comprising a hBNP promoter operably linked to a MTS and/or a gene that encodes a therapeutic, wherein the therapeutic is expressed in cardiomyocytes undergoing cardiac stress.

In some aspects, the therapeutic is predominantly expressed in the dysfunctional cardiomyocytes which are present in the right ventricle (RV) under cardiac disease during PH. In some aspects, predominantly expressed in the RV refers to at least 70%, 75%, 80%, 85%, 90%, 95% or 100% expression of the therapeutic in the RV.

In some aspects, the therapeutic is predominantly expressed in the mitochondrial fractionations of the cells in the right ventricle of the subject. In some aspects, predominantly expressed in the mitochondrial fractionation of the cells in the RV refers to at least 70%, 75%, 80%, 85%, 90%, 95% or 100% expression of the therapeutic in the Mito.

In some aspects, the therapeutic targets metabolic remodeling, mitochondrial dysfunction, and decreased fatty acid oxidation (FAO) in the right ventricle dysfunction. For example, HADHA overexpression can attenuate metabolic remodeling. In some aspects, HADHA overexpression can be an increase in expression to normal levels from a state of decreased expression.

In some aspects, the hBNP promoter becomes active under cardiac stress. Therefore, an active hBNP promoter drives expression of the MTS and/or the gene that encodes a therapeutic. BNP is produced and expressed in cardiomyocytes. Importantly, ventricular expression of the BNP is downregulated after birth and is strongly increased in adult ventricular cardiomyocytes under cardiac stress such as in PH. Therefore, a gene of interest that is driven by BNP promoter can lead to cardiomyocyte-specific expression and, importantly, has a low expression in non-stressed cardiomyocytes, but a strong expression in stressed cardiomyocytes. PH causes cardiac stress and therefore PH can activate expression from a BNP promoter.

In some aspects, disclosed is a hBNP promoter comprising the 5′ flanking region −1818 to +100 of DNA that is adjacent to the 5′ end of the hBNP gene and contains the promoter of the gene. In some aspects, the hBNP promoter is 1918 bps. In some aspects, hBNP promoter can be base pair 14 to 1931 of accession number U34833. Thus, in some aspects, the hBNP promoter comprises the sequence of SEQ ID NO:1.

In some aspects, altered promoter sequences can be used, wherein the altered promoter sequence retains promoter activity. In some aspect, an altered promoter sequence can be 70, 75, 80, 85, 90, 95, or 99% identical to SEQ ID NO:1. In some aspects, a functional fragment of the disclosed promoters can be used. A functional fragment can be any fragment of SEQ ID NO:1 that retains promoter activity.

In some aspects, the MTS encodes residues 1-40 of the amino acid sequence of very long chain acyl-CoA dehydrogenase. Thus, the MTS nucleic acid sequence can be

(SEQ ID NO: 2)
ATGCAGGCGGCTCGGATGGCCGCGAGCTTGGGGCGGCAGCTGCTGAGGC
TCGGGGGCGGAAGCTCGCGGCTCACGGCGCTCCTGGGGCAGCCCCGGCC
CGGCCCTGCCCGGCGGCCCTATGCCGGG.

In some aspects, the MTS operably linked to a cardiomyocyte-specific, cardiac stress-induced promoter is also linked to a gene of interest. In some aspects, the MTS is present at the 5′ end of a gene of interest.

In some aspects, the therapeutic is expressed in the right ventricle. In some aspects, the therapeutic is not expressed or is expressed significantly less in the left ventricle. In some aspects, the therapeutic is right ventricle specific meaning it is only expressed in the right ventricle or at least expressed at a much higher level in the right ventricle compared to the left ventricle. In some aspects, the hBNP promoter drives gene expression predominately in dysfunctional cardiomyocytes. In PH, where dysfunctional cardiomyocytes are present mostly in the right ventricle, gene expression is highly expressed in the right ventricle in comparison to left ventricle when under control of the hBNP promoter. In some aspects, there can be a very low expression level in normal cardiomyocytes in the left ventricle if the hBNP promoters has a low basal activity at baseline. In some aspects, there is expression in other major organs in the body besides the heart, such as a low expression in liver which is not surprising since hBNP promoter has some activity in liver.

In some aspects, the therapeutic can be, but are not limited to, hydroxyacyl-CoA dehydrogenase trifunctional multienzyme complex subunit alpha (HADHA) or Hydroxyacyl-CoA Dehydrogenase Trifunctional Multienzyme Complex Subunit Beta (HADHB). Example sequences of HADHA, can be, but are not limited to, (Human) NM_000182.5, (Rat) NM_130826.2, and (Mouse) NM_178878.3. Example sequences of HADHB, can be, but are not limited to, (Human) NM_000183.3, (Rat) NM_133618.3, (Mouse) NM_001289798.1, (Mouse) NM_001289799.1, and (Mouse) NM_145558.2

In some aspects, the therapeutic can be c-Src. Examples of c-Src can be, but are not limited to, (Human) NM_005417.5, (Human) NM_198291.3, (Rat) NM_031977.1, (Mouse) NM_001025395.2, and (Mouse) NM_009271.3.

In some aspects, the therapeutic can increase myocardial ATP generation, increase mitochondrial oxidative phosphorylation, increase cardiac contractility, increase myocyte health, reduce myocardial oxidative stress, reduce myocyte death/apoptosis, and/or decrease cardiac fibrosis.

In some aspects, the therapeutic is any therapeutic that treats RV dysfunction. IN some aspects, RV dysfunction can come from PH but can also be RV dysfunction associated with left ventricle, heart failure, cardiomyopathy, RV infarction, pulmonary valve stenosis, or tricuspid valve regurgitation.

In some aspects, the MTS is cleaved from the therapeutic after entering a mitochondria. In some aspects, there is a cleavage site between the PH therapeutic and the MTS.

In some aspects, there is no expression of the therapeutic in healthy cardiomyocytes. In some aspects, the hBNP promoter drives gene expression predominately in dysfunctional cardiomyocytes which are present mostly in the right ventricle during PH. In some aspects, there can be a very low expression level in normal cardiomyocytes in the left ventricle if the hBNP promoter has a low basal activity at baseline. In some aspects, there is expression in other major organs in the body besides the heart, such as a low expression in liver which is not surprising since hBNP promoter has some activity in liver.

In some aspects, the vector is administered via intravenous administration. In some aspects, the administration is via local injection, intracoronary, subcutaneous, intraperitoneal, or inhalation.

### 3. Increasing Fatty Acid Oxidation

Disclosed are methods of increasing fatty acid oxidation (FAO) and/or cardiolipin contents in a dysfunctional heart comprising administering a therapeutically effective amount of a vector to the subject,

wherein the vector comprises a nucleic acid sequence comprising a human pro-B-type natriuretic protein (hBNP) promoter operably linked to a mitochondrial targeting sequence (MTS) and a gene that encodes a therapeutic, wherein the therapeutic is expressed in cardiomyocytes undergoing cardiac stress. In some aspects, the therapeutic increases FAO and/or cardiolipin contents.

### G. Dosage

Disclosed are dosing regimens comprising administering a single dose of one or more of the disclosed compositions or vectors to a subject in need thereof, wherein the single dose comprises

5-13×10¹³ viral genomes/kg body weight.

Disclosed are dosing regimens comprising administering at least two doses of one or more of the disclosed compositions or vectors to a subject in need thereof, wherein each dose comprises

5-13×10¹³ viral genomes/kg body weight.

In some aspects, a single dose can be a continuous administration. In some aspects, a continuous administration can be hours, days, weeks, or months. In some aspects, there can be two or more doses. In some aspects, the two or more doses can be administered days, weeks, or months apart.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed nucleic acid sequences or vectors. The kits also can contain components of the disclosed nucleic acid sequences or vectors with instructions on how to create the disclosed nucleic acid sequences or vectors.

Disclosed herein are kits comprising one or more nucleic acid sequences comprising a cardiomyocyte-specific, cardiac stress-induced promoter operably linked to a MTS and/or a gene of interest.

Disclosed herein are kits comprising a vector comprising a nucleic acid sequence comprising a cardiomyocyte-specific, cardiac stress-induced promoter operably linked to a MTS and/or a gene of interest.

Disclosed herein are kits comprising a cell comprising a nucleic acid sequence comprising a cardiomyocyte-specific, cardiac stress-induced promoter operably linked to a MTS and/or a gene of interest.

### EXAMPLES

### A. Example 1: Targeting Mitochondrial Trifunctional Proteins to Improve Right Ventricular Function Using Gene Therapy

### 1. Background

Elevated pressure in the lung circulation or pulmonary hypertension (PH) is very prevalent in cardiopulmonary and metabolic diseases (e.g., heart failure with preserved or reduced ejection fraction, acute pulmonary embolism, COPD, sleep apnea, etc.) and is present in up to 50% of clinically indicated echocardiograms and 80% of right heart catheterizations. Severe PH is also seen in pulmonary arterial hypertension and chronic thromboembolic disease which are less common. Presence of PH is associated with poor prognosis irrespective of underlying conditions mostly related to the effect of high pressures on the right ventricle (RV). Presence RV dysfunction and failure in response to PH is an important prognostic marker and associated with poor functional tolerance, morbidity, and survival. Despite the known poor prognosis associated with RV dysfunction, to date, no RV targeted therapies are available, and the underlying mechanisms of RV dysfunction remain unclear. The salutary treatment strategies used in left ventricular dysfunction such as those targeting angiotensin II and beta-adrenergic receptors have failed to demonstrate any benefit in RV dysfunction. Thus, there is an unmet need to understand cellular and molecular mechanisms associated with RV dysfunction and to convert this knowledge into therapies.

Mitochondria (mito) through fatty acid oxidation (FAO) and oxidative phosphorylation (OXPHOS) are the major source of energy in the normal heart. Unmet energy needs due to mito dysfunction results in myocardial contractile failure, as seen in the dysfunctional RV. In settings of increased afterload such as in PH, RV undergoes metabolic reprogramming with a decrease in FAO and OXPHOS and an increase in glucose uptake, glycolysis and glutaminolysis. A fatty acid molecule such as palmitate can result in 105 ATP molecules after beta-oxidation and OXPHOS compared to 2 ATP per glucose molecule by glycolysis or 31 per glucose molecule through OXPHOS, albeit at a higher oxygen cost per ATP generation. While a decrease in FAO and OXPHOS has been traditionally viewed as an adaptive mechanism (especially in cases of ischemia mainly from coronary heart disease), recent studies also demonstrated that increasing FAO in dysfunctional heart and vasculature may be beneficial in certain settings such as diet induced cardiomyopathy and PH, where no overt ischemia is present. Most notably, treatment with a PPARγ agonist demonstrated improvement in PH and consequently RV function related to an improvement in FAO. However, PPARγ agonists cause worsening heart failure in patients and unlikely to be clinically used in settings of right heart failure, highlighting other off target effects of this drug therapy. Therefore, a strategy to directly improve FAO in heart is needed in settings of RV dysfunction and failure, which is evaluated in this study.

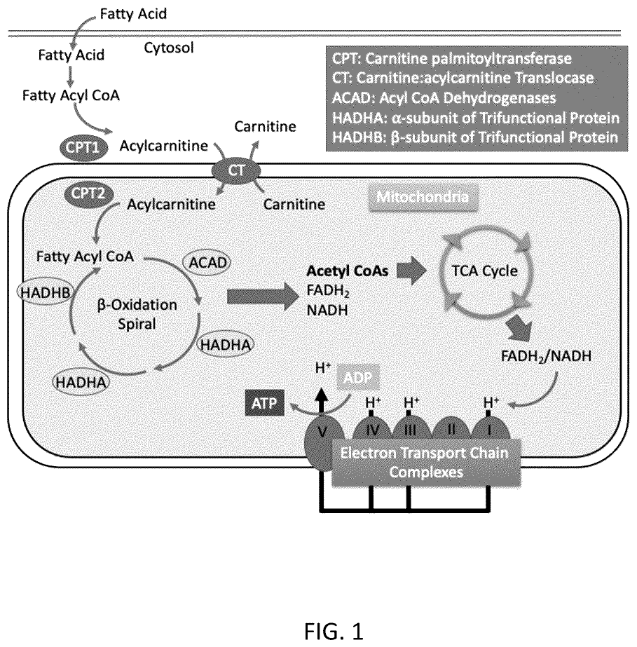

There are several transporters and enzymes involved in regulating fatty acid uptake and FAO in cardiomyocytes (FIG. 1). In the setting of PH, the expression of a number of these enzymes are decreased in RV myocardium that is associated with decreased FAO and RV function (see preliminary data below). Among them, CPT1 and 2 are important in transporting long chain (but not medium or small chain) fatty acids into the mito, and the trifunctional proteins (HADHA and HADHB) play a critical role in β-oxidation of fatty acids in the mito.

In addition, HADHA is important in mito structure, which is abnormal in settings of RV dysfunction and PH. Cardiolipin (CL) is a critical lipid in the mito and is important for mito structure and cristae organization. CL plays a critical role in assembly of electron transport chain complexes into structures known as supercomplexes (SCs) that increase the efficiency of the electron transport chain and reduce the production of reactive oxygen species. CL also increases electron chain activity and serves as a proton trap on the outer leaflet of inner mitochondrial membrane. The synthesis and maturation of CL involve many steps including a final step where acylation of monolysocardiolipin occurs and CL[18:1]₂[18:2]₂ is converted to mature CL[18:2]₄ catalyzed by α-subunit of HADHA. Therefore, the HADHA is critical to both FAO and mito bioenergetics (OXPHOS) as well as mito structure by facilitating mature CL. HADHA deficiency in rare genetic diseases is associated with cardiomyopathy, and reduction of HADHA expression in vitro showed abnormal mito structure and function. Therefore, the changes in mito function and structure in HADHA deficiency mirror those seen in RV mito in settings of PH, including smaller mito, abnormal cristae organization, and decrease in SC assembly. However, it is not known if increasing HADHA expression can result in improvement in mito structure and function, which will be investigated in this proposal.

While myocardial mito dysfunction has been identified in a number of cardiac diseases, including heart failure, ischemic and non-ischemic cardiomyopathy, diabetic cardiomyopathy and RV dysfunction, a therapeutic strategy targeting the mito has been elusive. Small molecule-based therapy lacks specificity and has off-target effects, and no mito protective drugs have passed clinical trials in large patient cohorts. In addition to small molecules, nanoparticle and liposomal delivery methods have also been tried with less favorable outcome, for which one possible explanation may have been poor delivery to the diseased cells and tissue. Therefore, in addition to identify a therapeutic target, there is a need to develop a novel reliable gene delivery strategy to modulate the gene-of-interest in only dysfunctional cardiomyocytes in diseases. Disclosed is an approach using gene therapy that demonstrates that the payload is delivered predominantly to dysfunctional cells.

### 2. Results

i. Preclinical Model of PH and RV Dysfunction:

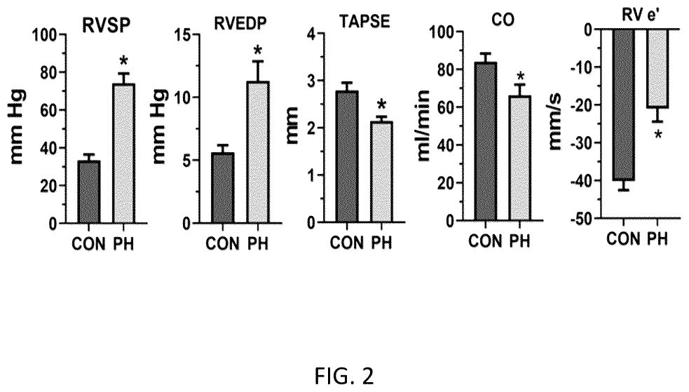

A preclinical PH rat model was used for the study. Adult Sprague Dawley (SD) rats receive 20 mg/kg sc SU5416 (a VEGF antagonist) are exposed to 3 wks of hypoxia (10% FiO2), followed by normoxia. Control rats injected with vehicle are kept in normoxia. At 7 wks time point, hemodynamic and echo assessments (FIG. 2) show PH (increased RV systolic pressure), RV systolic dysfunction (decreased TAPSE and CO) and diastolic dysfunction (decreased e′, elevated RVEDP) in PH rats in comparison to the control rats. No changes noted in LV.

ii. Decreased Mito FAO Respiration in RV Myocardium of PH Rats:

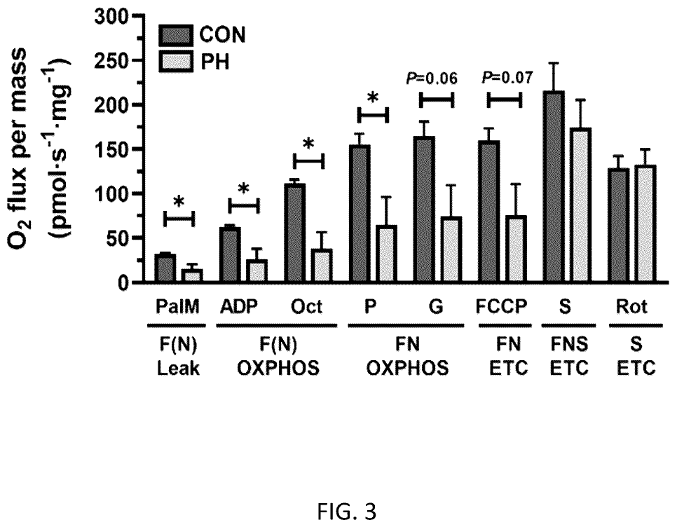

The mito FAO respiration was assessed in RV myocardium using high resolution 02K-FluoRespirometer (Oroboros). Oxygen consumption was recorded in permeabilized RV myocardial fibers from control and PH animals that have RV dysfunction (FIG. 2) to measure FAO-mediated maximal OXPHOS. FIG. 3 shows there is a significant decrease in ADP-induced, palmitoylcartinine- and octanoylcamitine-mediated maximal OXPHOS in mito from RV of the PH rats (7 wk) in comparison to controls, consistent with other reports and observations in humans. No such change was noted in the LV.

iii. Expression of Trifunctional Proteins (HADHA and HADHB) is Dramatically Decreased in RV Myocardium of PH Rats and in Dysfunctional RV of Human Patients:

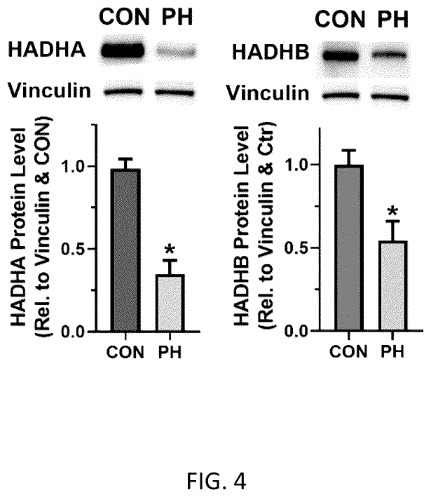

Proteomic profiles were assessed using mito isolated from RV of PH and controls rats (7 wk time point) and identified a significant decrease of a number of proteins that regulate FAO pathway. Among them, HADHA and HADHB are the most downregulated protein in the fatty oxidation pathway: both decreased by ˜70% in the RV of PH rats compared to that of control rats. To validate the proteomic findings, Western Blot was performed. Consistently, the data show a dramatic decrease in expression levels of HADHA and HADHB in the RV of PH rats (FIG. 4), but not in the LV (data not shown). We also found reduction of HADHA and HADHB expression in dysfunctional human RV than normal functional RV (data nor shown). Together, those data indicate that down-regulation of trifunctional proteins can play a key role in RV mito function and structure in settings of PH.

iv. Altered Mature CL, Mito Structure, and SC in RV Myocardium of PH Rats:

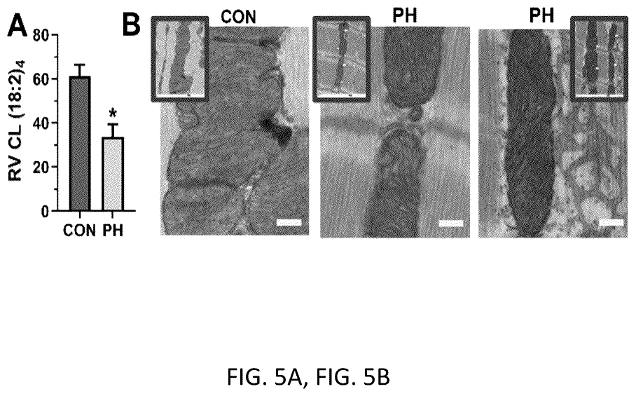

Given the importance of HADHA in mito cardiolipin maturation and subsequent mito structure regulation, whether mature CL and mito structure in RV myocardium of PH rats are altered was determined. Mature CL (CL[18:2]4) was measured by mass spectrometry. FIG. 5A shows significant reduction in the amount of mature CL in RV of PH rats, which is associated with decreased HADHA expression (FIG. 4).

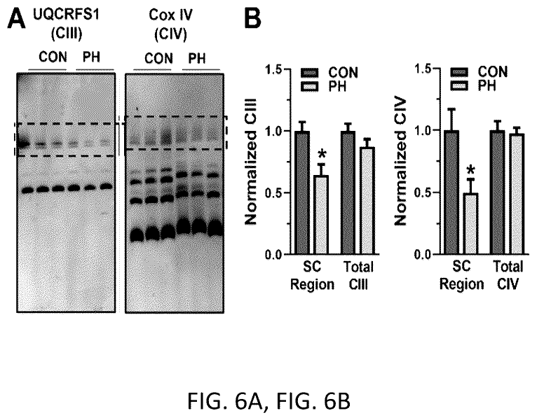

Mito structure was determined using transmission electron microscopy (TEM, FIG. 5B), which shows less dense cristae packing in the RV of PH rats compared to controls. In addition, SC assembly was assessed and the data show that the RV of PH rats have a decreased abundance of CIII and CIV in the supercomplex region (FIG. 6), while the total expression of either CIII or CIV is not changed. Together, these data indicate that decreased HADHA likely results in reduced mature CL, less dense cristae packing, and impaired SC assembly in RV myocardium of PH rats, which are sufficent to lead to mito dysfunction.

v. In Vitro Model Mimicking the Mito Changes Observed in PH Rat Model:

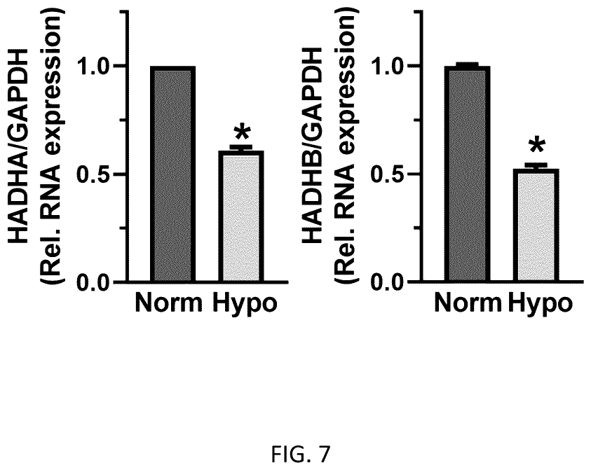

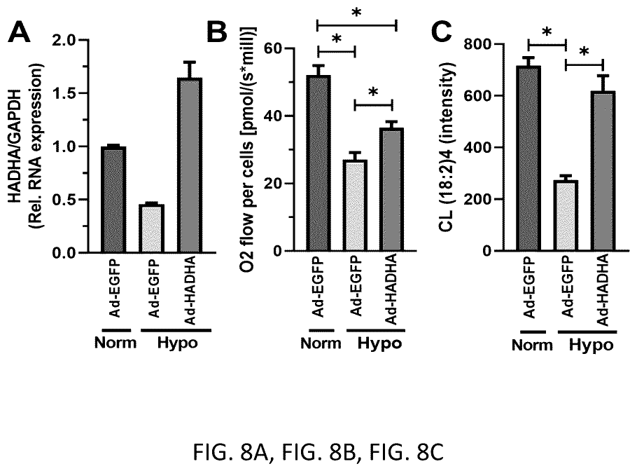

An in vitro model was generated to study the mechanisms and effect of decreased FAO proteins using a rat myocyte cell line (H9c2 cells) that is amenable to easy culture and genetic manipulations. Exposing the H9c2 cells to hypoxia (1% O₂) for 72 hrs recapitulated the expression profile observed in the dysfunctional RV of PH rats: a significant decrease in expression of several proteins regulating FAO including HADHA and HADHB (FIG. 7 and Table 1). In order to determine whether overexpression of HADHA can rescue the dysfunctional mito, adenovirus expressing HADHA were generated. As shown in FIG. 8A, the cells infected with adenovirus expressing HADHA have a 3.6-fold increase of HADHA expression than cells infected with adenovirus expressing EGFP (control) in hypoxia.

TABLE 1
mRNA levels of proteins regulating FAO in cells in hypoxia vs. cells in normoxia (set to 1)

| Gene name | HADHA | HADHB | CPT1A | CPT1B | CTP1C | CPT2 | ACADVL | ACADL | ACADM |
|---|---|---|---|---|---|---|---|---|---|
| Hypo vs | 0.61 | 0.52 | 0.51 | 0.73 | 0.54 | 0.59 | 0.52 | 0.48 | 0.80 |
| Norm, | | | | | | | | | |
| set to 1 | | | | | | | | | |

CPT1A, 1B, 1C, AND 2: carnitine palmitoyltransferase isoform 1A, 1B, 1C, and 2;
ACADVL, ACADL, ACADM: mitochondrial very long-chain, long-chain, and medium-chain specific acyl-CoA dehydrogenase

vi. Down-Regulation of HADHA in H9c2 Cells in Hypoxia is Associated with Decreased FAO and Mature CL, which can be Attenuated by HADHA Overexpression:

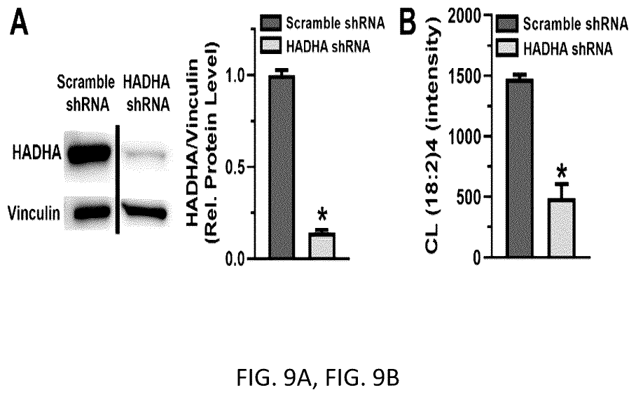

Whether H9c2 cells in hypoxia have altered FAO and mature CL was determined as well as whether overexpression of HADHA in H9c2 in hypoxia can attenuate the changes in FAO and mature CL. H9c2 cells were infected by adenovirus and kept in hypoxia for 72 hrs, followed by FAO assessments (Oroboros) or mature CL measurements (mass spectrometry). FIG. 8B shows a 50% reduction of FAO-linked respiration in hypoxic cells and HADHA expression is able to significantly improve FAO-linked respiration in hypoxia. Similarly, compared to normoxic cells, a 70% reduction in the account of mature CL in hypoxic cells was seen, which is almost fully attenuated by HADHA expression (FIG. 8C). We also found that HADHA specific knockdown is sufficient to decrease mature CL in H9c2 cells (FIG. 9). The data demonstrate that HADHA is critical in maintaining FAO and mito structure. In this proposal, the efficacy of TFP/HADHA overexpression in PH rat model is tested.

vii. A Novel Approach to Specifically Express Gene-of-Interest in Mito of Dysfunction RV Myocytes in PH:

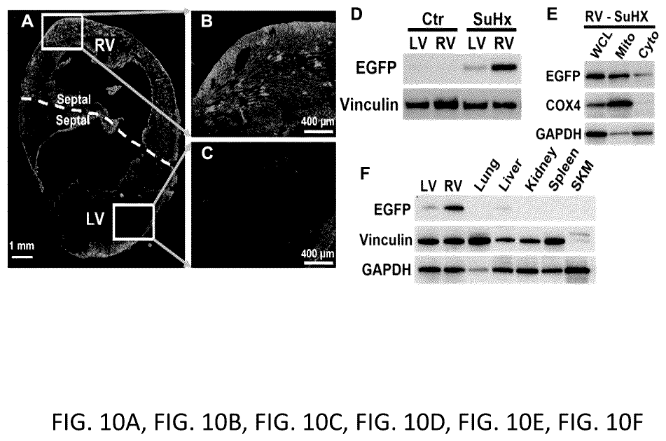

A previous study showed an increased expression of BNP in dysfunctional RV compared to normal RV in control animals and the LV in either control or PH rats. Based on this finding, the current approach using cardiotropic AAV9 and a human pro-B-type natriuretic protein (hBNP) promoter to drive gene expression predominantly in the mito of dysfunctional RV myocytes was developed. By combining with specific mito-targeted N-terminal presequences (MTS) with hBNP promoter (myocyte-specific in heart), this novel approach allows us to achieve cardiotropic AAV9 delivered, PH-induced, RV myocyte-specific, mito-targeted payload delivery in vivo, the data in FIGS. 10A-D show that EGFP is predominantly expressed in RV of the PH rat. The almost undetectable EGFP in control rat (FIG. 10D) indicates that hBNP promoter has little activity without stress. Importantly, the data show that the EGFP is mainly expressed in mito (FIG. 10E) using the MTS and in RV (FIG. 10F).

### B. Example 2: Design and Validation of Expression and Functional Effects of Mito Targeted TFP (HADHA, HADHB) Using Viral Vectors In Vitro

RV dysfunction in settings of PH is associated with poor outcomes irrespective of associated comorbidities and underlying conditions. However, there are no available therapies that are efficacious to treat RV dysfunction. Metabolic remodeling, mito dysfunction, and decreased FAO are hallmark pathophysiologic features associated with RV dysfunction and emerging evidence suggests that they may be therapeutically targeted. The current data demonstrate that there is a substantial decrease in trifunctional protein (HADHA and HADHB) expression in RV myocardium that is associated with RV dysfunction, abnormal RV FAO, decreased CL content, and abnormal mito cristae (FIG. 10). In vitro, overexpression of HADHA improved mito FAO and mature CL content despite hypoxia exposure, demonstrating that HADHA overexpression is sufficient to attenuate the metabolic remodeling. Furthermore, a strategy to in vivo deliver mito targeted proteins in stressed cardiomyocytes only has been developed. The overall objective of this proposal is to develop a gene therapy based therapeutic approach to facilitate FAO and preserve mito structure by restoring the expression of TFP in dysfunctional RV and thereby consequently improve RV function in settings of PH. Reduced expression of TFP results in impaired myocardial energetics and mito structure that collectively cause RV dysfunction and failure. Therefore, restoring TFP in dysfunctional RV myocytes can result in improvement of RV function

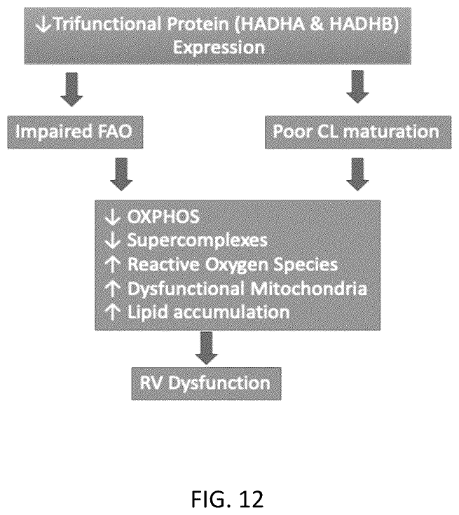

Generate the following viral vectors for in vitro and in vivo studies: (i) Adenovirus with MTS-huHADHA, (ii) Adenovirus with MTS-huHADHB, (iii) Adenovirus with MTS-huAHDHA-huHADHB, (iv) Replace EGFP in our current AAV9 construct (FIG. 12) with huHADHA or (v) huHADHB and generate hBNP-MTS-HADHA-AAV9 and hBNP-MTS-HADHB-AAV9, respectively, and (vi) control viruses: Ad-MTS-EGFP, Ad-hBNP-MTS-EGFP.

Optimize the adenoviral infection strategy using isolated adult rat cardiomyocytes under normoxia/hypoxia in vitro and assess mito specific exogenous gene expression using mito fractionation and immunoblot, mito FAO using Oroboros, mito SC abundance/activity using blue native page, mature CL content using mass spectrometry, and mito structure using TEM.

Optimize the AAV infection strategy using isolated adult rat cardiomyocytes under vehicle/phenylephrine in vitro and determine mito specific exogenous gene expression using mito fractionation and immunoblot, mito FAO using Oroboros, mito SC abundance/activity using blue native page, mature CL content using mass spectrometry, and mito structure using TEM.

Determine the efficiency and efficacy of AAV9-mediated gene delivery in dysfunctional RV in preclinical rat PH model on RV mito structure/function, metabolism, and RV function in vivo.

This proposal demonstrates that metabolism in dysfunctional RV can be selectively targeted by gene therapy in PH, which in turn results in improved metabolism and mito structure and function, and improved RV function. A gene therapy approach to specifically target mito in dysfunctional RV Cardiomyocytes can be used.

i. Research Design

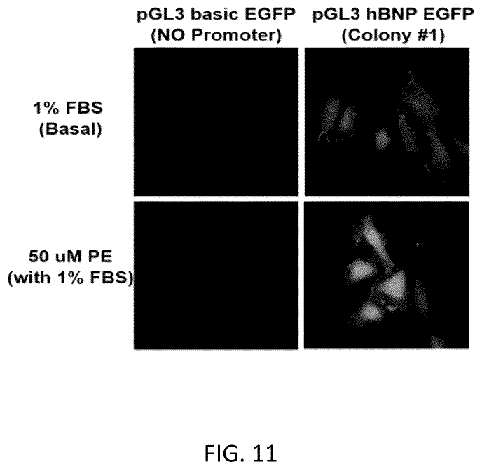

Experimental Models: Isolated adult cardiomyocytes are used for in vitro studies as metabolic profile of neonatal cells and cell lines do not fully recapitulate that of mature adult cardiomyocytes to first validate the functional effects of the viral constructs. Two in vitro models can be used: (i) exposure to 1% hypoxia for 48-72 h (FIG. 8) that results in decreased FAO and CL similar to what is observed in dysfunctional RV and (ii) exposure to vehicle or phenylephrine (10-50 uM) for 48-72 h to stimulate BNP promoter (FIG. 11). For in vivo studies, an established preclinical rat PH model and RV dysfunction will be used (FIG. 2).

ii. Design and Validate Expression and Functional Effects of Mito Targeted TFP (HADHA, HADHB) Using Viral Vectors In Vitro.

Mito TFP catalyzes beta-oxidation of fatty acids, and the a-subunit (HADHA) also catalyzes maturation of CL, a critical mito lipid to maintain mito structure and function. The data show that the expression levels of HADHA and HADHB are substantially reduced in the dysfunctional RV in PH and are associated with impaired FAO, CL, abnormal cristae, and decreased supercomplexes. The data also demonstrate that increasing HADHA expression alone is sufficient to increase mature CL levels and partially recover FAO in settings of hypoxia in culture.

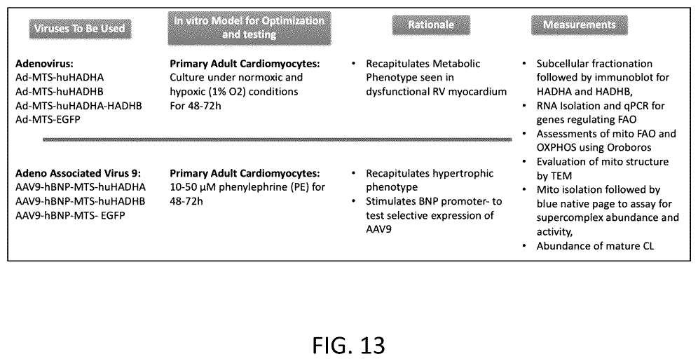

Viral vectors can be generated for in vitro and in vivo studies. The in vitro assays can be validated using freshly isolated primary adult cardiomyocytes under two separate conditions of stress: hypoxia and phenylephrine. While hypoxia recapitulated the metabolic phenotype of failing RV, phenylephrine stimulation is associated with activation of BNP and is a pathophysiologically relevant in vitro model as well. Therefore, adenovirus is first used to study the effect of HADHA alone, HADHB alone, and combination of HADHA and HADHB in settings of hypoxia to ascertain the best infection strategy to recover the metabolic phenotype and mito structure and function. In parallel, the Cardiomyocytes treated with phenylephrine and infected with AAV9 expressing hBNP promoter-driven HADHA or HADHB are used to ascertain expression levels, subcellular localization, and functional effects of expressing HADHA, HADHB and both in phenylephrine-treated cells compared to controls. These studies provide the in vitro validation of these constructs and help with deciding the best strategy of infection for in vivo experiments (FIG. 13).

Generate the following viral vectors for in vitro and in vivo studies: (i) Adenovirus with MTS-huHADHA (ii) Adenovirus with MTS-huHADHB (iii) Adenovirus with MTS-huAHDHA-huHADHB (iv) Replace EGFP in our current AAV9 construct (FIG. 10) with huHADHA or (v) huHADHB and generate hBNP-MTS-HADHA-AAV9 and hBNP-MTS-HADHB-AAV9, respectively, and (vi) control viruses: Ad-MTS-EGFP, AAV9-hBNP-MTS-EGFP.

Since the payload in AAV9 is limited to ˜4.7 kb, both HADHA and HADHB (combined size ˜5 kB without the MTS and hBNP promoter) may not be able to be packaged together in the same AAV9.

Optimize the adenoviral infection strategy using isolated adult rat cardiomyocytes under normoxia/hypoxia in vitro and assess mito specific exogenous gene expression using mito fractionation and immunoblot, mito FAO using Oroboros, mito SC abundance/activity using blue native page, mature CL content using mass spectrometry, and mito structure using TEM.

Primary cardiomyocytes are isolated from adult male and female SD rats. The cells are cultured in either normoxic or hypoxic (1% O₂) conditions for 48-72 h after infection with the following: Ad-EGFP, Ad-MTS-HADHA, Ad-MTS-HADHB, and Ad-MTS-HADHA-HADHB (as in FIG. 8A). At the end of the experiments, myocytes are collected for the following assays: (i) Subcellular fractionation followed by immunoblot for HADHA and HADHB, (ii) RNA Isolation and qPCR for genes regulating FAO (e.g. Table 1), (iii) Assessments of mito FAO and OXPHOS using Oroboros (see FIG. 8B), (iv) Evaluation of mito structure by TEM, (v) mito isolation followed by blue native page to assay for supercomplex abundance and activity, and (vi) abundance of mature CL and precursors using MALDI-TOF or LCMS (FIG. 8C).

Optimize the AAV infection strategy using isolated adult rat cardiomyocytes under vehicle/phenylephrine in vitro and determine mito specific exogenous gene expression using mito fractionation and immunoblot, mito FAO using Oroboros, mito SC abundance/activity using blue native page, mature CL content using mass spectrometry, and mito structure using TEM.

Primary cardiomyocytes from adult male and female SD rats are cultured either in presence or absence of 10-50 μM phenylephrine (a strong myocyte hypertrophy agonist that stimulates hBNP promoter, FIG. 11) for 48-72 h after infection with the following: AAV9-hBNP-MTS-EGFP, AAV9-hBNP-MTS-HADHA, AAV9-hBNP-MTS-HADHB or both AAV9-hBNP-MTS-HADHA and AAV9-hBNP-MTS-HADHB. At the end of the experiments, myocytes are collected for the following assays: (i) Subcellular fractionation followed by immunoblot for HADHA and HADHB, (ii) RNA Isolation and qPCR for genes regulating FAO (e.g. Table 1), (iii) Assessments of mito FAO and OXPHOS using Oroboros (see FIG. 8B), (iv) Evaluation of mito structure by TEM, (v) mito isolation followed by blue native page to assay for supercomplex abundance and activity, and (vi) abundance of mature CL and precursors using MALDI-TOF or LCMS (FIG. 8C).

Analytical Plan: For in vitro experiments, samples from 5-6 cell batches or hearts per group are examined. The infection with the viruses increases the mito expression of the respective proteins is confirmed. The mRNA expression of other FAO related proteins is assessed to evaluate for any cellular adaptation in response to overexpression of target proteins. FAO and OXPHOS are evaluated as in FIG. 8B, palmitate-mediated maximal oxygen consumption in presence of FCCP that is subsequently suppressed with etomoxir (Eto), a β-oxidation inhibitor-specifically inhibits CPT1, will be used to measure FAO-linked respiration capacity. Cristae packing can be evaluated both qualitatively and by measurement of distance between cristae on high resolution TEM images, and finally SC abundance and activity is measured as in FIG. 6. The groups are compared using ANOVA with post hoc test for multiple comparisons. A p value <0.05 will be considered significant.

Hypoxia and chronic phenylephrine treatment results in decreased FAO, reduced expression of FAO related proteins, increased distance between cristae and loosely packed mito, decreased mature CL, and reduced SC abundance and/or activity. HADHA overexpression is associated with improved FAO, mature CL contents, mito structure, and SC abundance/activity in comparison to controls (cells transfected with EGFP). HADHB has an overall mild effect, but could show a good synergistic effect when co-expressed with HADHA. The AAV9-delivered, hBNP promoter-driven HADHA and/or HADHB is overexpressed in cells treated with phenylephrine but not in control cells treated with vehicle.

### C. Example 3: Determine the Efficiency and Efficacy of AAV9-Mediated Gene Delivery in Dysfunctional RV in Preclinical Rat PH Model on RV Mito Structure/Function, Metabolism, and RV Function In Vivo

Adult rats subjected to sc SU5416 (a VEGF antagonist) are exposed to 3 wks of hypoxia (10% FiO₂), followed by normoxia, to induce PH. The rats develop severe PH and RV dysfunction and will be used at 7 wk timepoint (FIG. 2). Control rats injected with vehicle are kept in normoxia.

In vivo optimization studies: AAV9 vectors generated can be injected and tested (AAV9 expressing hBNP-MTS-EGFP as control) 2 days before PH induction and assess expression of HADHA and HADHB and EGFP. Dose optimization is performed by injecting different amount of AAV9 (5-13×10¹³ genomes/kg body weight) to restore the HADHA and HADHB expression level comparable to the endogenous level in control rats. Cardiac cells (e.g., cardiomyocytes, fibroblasts, endothelial cells, smooth muscle cells) are isolated and used in immunoblots to determine the RV cardiomyocyte-specific expression. Next, expression of EGFP and HADHA/HADHB at 1, 4, 7, 10 wks is assessed to ascertain the temporal expression profile in the RV using the optimal dose. This informs the use of the right timing of the AAV9 injection for expression of the target molecules. The expression of AAV9 mediated genes can alter the levels of target proteins in the heart by ˜3 wks post PH induction. At 3 wks time point, the PH and RV dysfunction is already present.

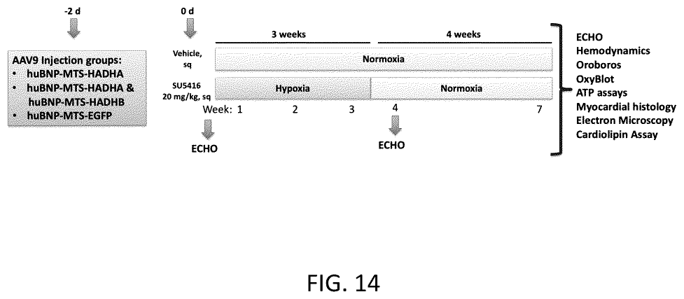

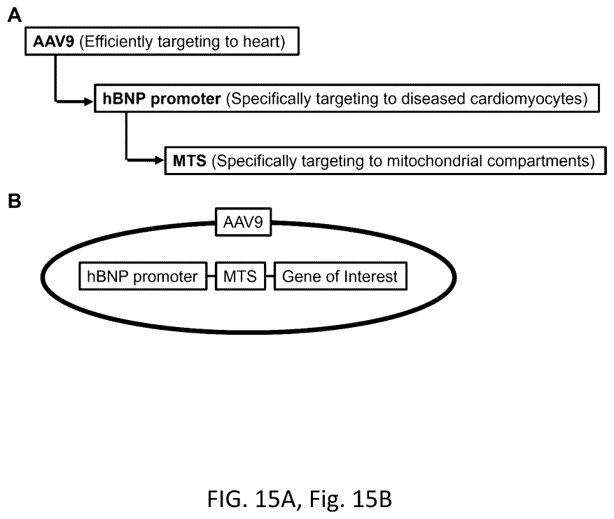

Efficacy Studies: After optimizing and validation of mito-targeted HADHA and HADHB expression in RV, effects of restoring mito HADHA and HADHB expression on RV are determined in PH using groups: Treatment Group: PH rats with AAV9-mediated, hBNP promoter-driven MTS-HADHA and/or MTS-HADHB expression, and Control Group: PH rats with AAV9-mediated, hBNP promoter-driven EGFP expression (FIG. 14). At 7 wk time point, the following are assessed: (i) Cardiac morphology and function assessed by serial echocardiography (also before AAV injection and 4 weeks after PH induction). (ii) Invasive hemodynamics at the end of the experiments to assess RV systolic and diastolic pressures. (iii) Oroboros for RV mito respirations with FAO and mito ROS in RV myocardium. (Iv) OxyBlot to assess oxidative stress and ATP assays to measure ATP amount in RV myocardium. (v) Myocardial histology (gross morphology, H&E, and TUNEL staining for apoptosis, trichrome staining for fibrosis), (iv) EM for assessment of mito size and cristae density, and (vii) levels of mature CL.

Analytical Plan: Both sexes are used for all experiments and analyses is performed separately for each sex. Blinding: Personnel performing assessments of histology, echocardiography and hemodynamics is blinded to the experimental conditions. Power Analyses: TAPSE and RV e′ are used as endpoints for systolic and diastolic function, which have a standard deviation of 0.34 mm and 12.3 mm/s, respectively. In PH animals, they are reduced by 0.65 mm and 19 mm/s, respectively. With 12 rats per group per sex, there is 80% power to see a 0.4 mm increase in TAPSE, and 14.7 mm/s improvement in RV e′ using two-tailed t-test with an alpha of 0.05. Statistical Analyses: Unpaired Student's t-tests or ANOVA with post hoc test is used to compare 2 or more than 2 groups, respectively. Nonparametric tests are performed if data is not normally distributed. A P value of <0.05 is considered statistically significant.

An optimal dose of AAV9 to see an improvement in RV function in the animals infected with AAV9 expressing HADHA or both HADHA and HADHB can be achieved. The improved function is associated with improvement in FAO, decreased ROS, increase in myocardial ATP, less fibrosis and apoptosis, and improvement in mito structure/cristae density and mature CL amount. No change in pulmonary artery pressures between the groups.

Those skilled in the art will recognize, or be able to ascertain using no more than routine experimentation, many equivalents to the specific embodiments of the method and compositions described herein. Such equivalents are intended to be encompassed by the following claims.

### REFERENCES

1 Clements, R. T. et al. Treatment of Pulmonary Hypertension With Angiotensin II Receptor Blocker and Neprilysin Inhibitor Sacubitril/Valsartan. Circ Heart Fail 12, e005819, doi:10.1161/CIRCHEARTFAILURE.119.005819 (2019).
2 Casserly, B. et al. C-type natriuretic peptide does not attenuate the development of pulmonary hypertension caused by hypoxia and VEGF receptor blockade. Life Sci 89, 460-466, doi:10.1016/j.lfs.2011.07.009 (2011).
3 Suen, C. M., Chaudhary, K. R., Deng, Y., Jiang, B. & Stewart, D. J. Fischer rats exhibit maladaptive structural and molecular right ventricular remodelling in severe pulmonary hypertension: a genetically prone model for right heart failure. Cardiovasc Res 115, 788-799, doi:10.1093/cvr/cvy258 (2019).
4 Jankowich, M., Maron, B. A. & Choudhary, G. Mildly elevated pulmonary artery systolic pressure on echocardiography: bridging the gap in current guidelines. Lancet Respir Med 9, 1185-1191, doi:10.1016/52213-2600(21)00072-2 (2021).
5. Vonk Noordegraaf, A. & Galie, N. The role of the right ventricle in pulmonary arterial hypertension. Eur Respir Rev 20, 243-253, doi:10.1183/09059180.00006511 (2011).
6 Ryan, J. J. & Archer, S. L. The right ventricle in pulmonary arterial hypertension: disorders of metabolism, angiogenesis and adrenergic signaling in right ventricular failure. Circ Res 115, 176-188, doi:10.1161/CIRCRESAHA.113.301129 (2014).
7 Prisco, S. Z., Thenappan, T. & Prins, K. W. Treatment Targets for Right Ventricular Dysfunction in Pulmonary Arterial Hypertension. JACC Basic Transl Sci 5, 1244-1260, doi:10.1016/j.jacbts.2020.07.011 (2020).
8 Leopold, J. A. et al. Diagnosis and Treatment of Right Heart Failure in Pulmonary Vascular Diseases: A National Heart, Lung, and Blood Institute Workshop. Circ Heart Fail 14, doi:1161/CIRCHEARTFAILURE.120.007975 (2021).
9 Ventura-Clapier, R., Gamier, A., Veksler, V. & Joubert, F. Bioenergetics of the failing heart. Biochim Biophys Acta 1813, 1360-1372, doi:10.1016/j.bbamcr.2010.09.006 (2011).
10. Zhou, B. & Tian, R. Mitochondrial dysfunction in pathophysiology of heart failure. J Clin Invest 128, 3716-3726, doi:10.1172/JCI120849 (2018).
11 Culley, M. K. & Chan, S. Y. Mitochondrial metabolism in pulmonary hypertension: beyond mountains there are mountains. J Clin Invest 128, 3704-3715, doi:10.1172/JCI120847 (2018).
12 Asosingh, K. & Erzurum, S. Mechanisms of right heart disease in pulmonary hypertension (2017 Grover Conference Series). Pulm Circ 8, 2045893217753121, doi:10.1177/2045893217753121 (2018).
13 Legchenko, E. et al. PPARgamma agonist pioglitazone reverses pulmonary hypertension and prevents right heart failure via fatty acid oxidation. Sci Transl Med 10, doi:10.1126/scitranslmed.aao0303 (2018).
14 Gomez-Arroyo, J. et al. Metabolic gene remodeling and mitochondrial dysfunction in failing right ventricular hypertrophy secondary to pulmonary arterial hypertension. Circ Heart Fail 6, 136-144, doi:10.1161/CIRCHEARTFAILURE.111.966127 (2013).
15. Piao, L. et al. Cardiac glutaminolysis: a maladaptive cancer metabolism pathway in the right ventricle in pulmonary hypertension. J Mol Med (Berl) 91, 1185-1197, doi:10.1007/s00109-013-1064-7 (2013).
16 Koop, A. C. et al. Metabolic Remodeling in the Pressure-Loaded Right Ventricle: Shifts in Glucose and Fatty Acid Metabolism-A Systematic Review and Meta-Analysis. J Am Heart Assoc 8, e012086, doi:10.1161/JAHA.119.012086 (2019).
17 Shao, D. et al. Increasing Fatty Acid Oxidation Prevents High-Fat Diet-Induced Cardiomyopathy Through Regulating Parkin-Mediated Mitophagy. Circulation 142, 983-997, doi:10.1161/CIRCULATIONAHA.119.043319 (2020).
18 Sinha, B. & Ghosal, S. Assessing the need for pioglitazone in the treatment of patients with type 2 diabetes: a meta-analysis of its risks and benefits from prospective trials. Sci Rep 10, 15781, doi:10.1038/s41598-020-72967-8 (2020).
19 Lopaschuk, G. D., Ussher, J. R., Folmes, C. D., Jaswal, J. S. & Stanley, W. C. Myocardial fatty acid metabolism in health and disease. Physiol Rev 90, 207-258, doi:10.1152/physrev.00015.2009 (2010).
20. Lu, Y. W. & Claypool, S. M. Disorders of phospholipid metabolism: an emerging class of mitochondrial disease due to defects in nuclear genes. Front Genet 6, 3, doi:10.3389/fgene.2015.00003 (2015).
21 Miklas, J. W. et al. TFPa/HADHA is required for fatty acid beta-oxidation and cardiolipin re-modeling in human cardiomyocytes. Nat Commun 10, 4671, doi:10.1038/s41467-019-12482-1 (2019).
22 Taylor, W. A. et al. Human trifunctional protein alpha links cardiolipin remodeling to beta-oxidation. PLoS One 7, e48628, doi:10.1371/joumal.pone.0048628 (2012).
23 Forini, F., Canale, P., Nicolini, G. & Iervasi, G. Mitochondria-Targeted Drug Delivery in Cardiovascular Disease: A Long Road to Nano-Cardio Medicine. Pharmaceutics 12, doi:10.3390/pharmaceutics12111122 (2020).
24 Toba, M. et al. Temporal hemodynamic and histological progression in Sugen5416/hypoxia/normoxia-exposed pulmonary arterial hypertensive rats. Am J Physiol Heart Circ Physiol 306, H243-250, doi:10.1152/ajpheart.00728.2013 (2014).
25. Vang, A. et al. alpha7 Nicotinic acetylcholine receptor mediates right ventricular fibrosis and diastolic dysfunction in pulmonary hypertension. JCI Insight 6, doi:10.1172/jci.insight.142945 (2021).
26 Ritterhoff, J. et al. Metabolic Remodeling Promotes Cardiac Hypertrophy by Directing Glucose to Aspartate Biosynthesis. Circ Res 126, 182-196, doi:10.1161/CIRCRESAHA.119.315483 (2020).
27 Domenger, C. & Grimm, D. Next-generation AAV vectors-do not judge a virus (only) by its cover. Hum Mol Genet 28, R3-R14, doi:10.1093/hmg/ddzl48 (2019).

1. A nucleic acid sequence comprising a human pro-B-type natriuretic protein (hBNP) promoter operably linked to a mitochondrial targeting sequence (MTS) and/or a gene of interest.

2. The nucleic acid sequence of claim 1, wherein the MTS is present at the 5′ end of the gene of interest.

3. The nucleic acid sequence of claim 1, wherein the MTS is
(SEQ ID NO: 2)
ATGCAGGCGGCTCGGATGGCCGCGAGCTTGGGGCGGCAGCTGCTGAGGC
TCGGGGGCGGAAGCTCGCGGCTCACGGCGCTCCTGGGGCAGCCCCGGCC
CGGCCCTGCCCGGCGGCCCTATGCCGGG.

4. The nucleic acid sequence of claim 1, wherein the gene of interest is a gene that encodes a pulmonary hypertension (PH) therapeutic.

5. The nucleic acid sequence of claim 4, wherein the PH therapeutic is hydroxyacyl-CoA dehydrogenase trifunctional multienzyme complex subunit alpha (HADHA), Hydroxyacyl-CoA Dehydrogenase Trifunctional Multienzyme Complex Subunit Beta (HADHB), or cSrc.

6. The nucleic acid sequence of claim 1, wherein the hBNP promoter comprises the sequence of
(SEQ ID NO: 1)
GTAGAAACACCTTGTGATCACCCTGGCAGTGATTATGAGCTTCAGGTCTGGAATCA
GACTGCTGGCTAGACTAATCAGACTGGTTAGAATCCAGGATTTATCATGTGTCAATT
GTGTGACTTTTGGAAAGTAGATTAATTCATGAACACCATTTCCTCCTCTGAAGTGAG
GAATAATAACCGTGCTTTTCTCACCTCAGGGGCAGATGCTATTTTTTAGGCAAGATC
TGCTTAGAGGTCCCAGTTTCTTATTGCTGCCCTTCTCTGCTGTAACTCTTCTCCCCTC
ATAGACAGCTCCACTCCTCCAGCCTGCTGCTTGTTGACACCAATTCTCTGGAAGGGG
AGTGACATCAGTCATATATGCTTTAGGGGGGTATTTAAGCTGCTATGACTCTTCTCA
GGGGCATTTCTCTCCAAAGTCTCACTTCTAATCACCAGGCCACCTGCTAATGATAAT
TAGATCATGGGTGGTCAGATGAAGGAGGCACTGGGAGAGGGGAAATCCCCATATCT
CTGGTATCCCAGGAAATAGATAACCATCATTCCAGCCATCCTTTTGTTTTCTTTCTTT
CTTTCTTTCTTTCTTACTTTCTTTCTTTCTTTCTTTCCTTCTCTCTTTCTTCCTTCCGCTC
TCTCTCTGTCAACCAGGCTGGAGTGCAGTGGCGTGATCTCAGCTCACTGCAACCTCC
ACCTCCTGGGTTCAAGTGATTCTCCTTCCTCAGCCTCCCGAGTAGCTGGGACTACAG
GCGCCTGCCACCATGCCCAGCTAATTTTTGGTAATTTTAGTAGAGACGGGGTTTCAC
CGTGGTCTCGATCTCCTGACCTCGTGATCCGACCGCCTCGGCCTCTCAAAGTGCTGG
GATTACAGGCGTGAACCACCATGCCCAGCCTATCCTTTTGTTTTCCATCCTGTGTTG
GCTTGGTGGGGGAGAGGAGGTGTTGACACGTGGAGGACACACATATAAGGCATTCT
TGGGTGACTTCGTCATCACTGGACCCTATCTCTCAAAATTCCAGCGAAATCTGCTCT
TCCCTTTAAGGAGTGAAAGAAGGGTCAGCATTCCAGAAGTTCCTGGTCATACCCAG
GCTTTTAATGAATTGCCACTGGGGAATCAGCATCCCGTTGCTGTAAGGACTATAAGA
TGGCGGATTGTGAGAGCATAGGGAAAGGTCTCGGAGGTCTCTTGTCCTTGCTCCAC
GCAGGTCTTTCTGGCCTGAAAATCCCGTTGAAGAGAGCAGCTCTTGAGAGTTTGCTC
CAAGTTCCCTCGGGGTGATCAGCACCACGGACAGGGGCCAGGGCGCCCCCGAGGAC
CCGCAGGCAGGCAGGGTGCACAGCGGGGAGCAGGTGCTGCGCTACGTGCGGGCCA
GGGAACTCGCGCGGGGAGGGGAGAGGCGCCGCGGGTGGCGGGGTCTTGGCCGGGG
CTGTTTTCGCTGTGAGATCACCCCGTGCTCCCAGCGCTCACGTCGGTCCTCGGAAAG
CCGGGGTCCTCCCTGCCTTTTCCAGCAACGGTGGGGTGGGGAGGCAGGAAGAAAGC
GCCAACCTAGGACCCCGGAGATTTGCAGCAAAGGAAGAAGCGGGAGACGGGCACT
TGTCTGTGTCTCCAGCGCGTTCCTGCCCCCCGCCGACCCGGCCCATTTCTATACAAG
GTCGCTCTGCCCGGTCTCCACCTCCCACGTGCAGGCCGCGGAGGGGCTCATTCCCGG
GCCCTGATCTCAGAGGCCCGGAATGTGGCTGATAAATCAGAGACTAGACCTGCATG
GCAGGCAGGCCCGACACTCAGCTCCAGGATAAAAGGCCACGGTGTCCCGAGGAGC
CAGGAGGAGCACCCCGCAGGCTGAGGGCAGGTGGGAAGCAAACCCGGACGCATCG
CAGCAGCAGCAGCAGCAGCAGAAGCAGCAGCAGCAGCCTCCGCAGTCCC

7. A vector comprising the nucleic acid sequence of claim 1.

8. The vector of claim 7, wherein the vector is a viral vector.

9. The vector of claim 8, wherein the viral vector is an adenoviral associated vector (AAV).

10. The vector of claim 9, wherein the adenoviral associated vector is AAV9.

11. A method of treating right ventricle (RV) dysfunction and failure in pulmonary hypertension (PH) in a subject comprising administering a therapeutically effective amount of a vector to the subject,
wherein the vector comprises a nucleic acid sequence comprising a human pro-B-type natriuretic protein (hBNP) promoter operably linked to a mitochondrial targeting sequence (MTS) and/or a gene that encodes a PH therapeutic,
wherein the PH therapeutic is expressed in cardiomyocytes undergoing cardiac stress.

12. The method of claim 11, wherein the hBNP promoter becomes active under cardiac stress.

13. The method of claim 11, wherein the hBNP promoter comprises the sequence of
(SEQ ID NO: 1)
GTAGAAACACCTTGTGATCACCCTGGCAGTGATTATGAGCTTCAGGTCTGGAATCA
GACTGCTGGCTAGACTAATCAGACTGGTTAGAATCCAGGATTTATCATGTGTCAATT
GTGTGACTTTTGGAAAGTAGATTAATTCATGAACACCATTTCCTCCTCTGAAGTGAG
GAATAATAACCGTGCTTTTCTCACCTCAGGGGCAGATGCTATTTTTTAGGCAAGATC
TGCTTAGAGGTCCCAGTTTCTTATTGCTGCCCTTCTCTGCTGTAACTCTTCTCCCCTC
ATAGACAGCTCCACTCCTCCAGCCTGCTGCTTGTTGACACCAATTCTCTGGAAGGGG
AGTGACATCAGTCATATATGCTTTAGGGGGGTATTTAAGCTGCTATGACTCTTCTCA
GGGGCATTTCTCTCCAAAGTCTCACTTCTAATCACCAGGCCACCTGCTAATGATAAT
TAGATCATGGGTGGTCAGATGAAGGAGGCACTGGGAGAGGGGAAATCCCCATATCT
CTGGTATCCCAGGAAATAGATAACCATCATTCCAGCCATCCTTTTGTTTTCTTTCTTT
CTTTCTTTCTTTCTTACTTTCTTTCTTTCTTTCTTTCCTTCTCTCTTTCTTCCTTCCGCTC
TCTCTCTGTCAACCAGGCTGGAGTGCAGTGGCGTGATCTCAGCTCACTGCAACCTCC
ACCTCCTGGGTTCAAGTGATTCTCCTTCCTCAGCCTCCCGAGTAGCTGGGACTACAG
GCGCCTGCCACCATGCCCAGCTAATTTTTGGTAATTTTAGTAGAGACGGGGTTTCAC
CGTGGTCTCGATCTCCTGACCTCGTGATCCGACCGCCTCGGCCTCTCAAAGTGCTGG
GATTACAGGCGTGAACCACCATGCCCAGCCTATCCTTTTGTTTTCCATCCTGTGTTG
GCTTGGTGGGGGAGAGGAGGTGTTGACACGTGGAGGACACACATATAAGGCATTCT
TGGGTGACTTCGTCATCACTGGACCCTATCTCTCAAAATTCCAGCGAAATCTGCTCT
TCCCTTTAAGGAGTGAAAGAAGGGTCAGCATTCCAGAAGTTCCTGGTCATACCCAG
GCTTTTAATGAATTGCCACTGGGGAATCAGCATCCCGTTGCTGTAAGGACTATAAGA
TGGCGGATTGTGAGAGCATAGGGAAAGGTCTCGGAGGTCTCTTGTCCTTGCTCCAC
GCAGGTCTTTCTGGCCTGAAAATCCCGTTGAAGAGAGCAGCTCTTGAGAGTTTGCTC
CAAGTTCCCTCGGGGTGATCAGCACCACGGACAGGGGCCAGGGCGCCCCCGAGGAC
CCGCAGGCAGGCAGGGTGCACAGCGGGGAGCAGGTGCTGCGCTACGTGCGGGCCA
GGGAACTCGCGCGGGGAGGGGAGAGGCGCCGCGGGTGGCGGGGTCTTGGCCGGGG
CTGTTTTCGCTGTGAGATCACCCCGTGCTCCCAGCGCTCACGTCGGTCCTCGGAAAG
CCGGGGTCCTCCCTGCCTTTTCCAGCAACGGTGGGGTGGGGAGGCAGGAAGAAAGC
GCCAACCTAGGACCCCGGAGATTTGCAGCAAAGGAAGAAGCGGGAGACGGGCACT
TGTCTGTGTCTCCAGCGCGTTCCTGCCCCCCGCCGACCCGGCCCATTTCTATACAAG
GTCGCTCTGCCCGGTCTCCACCTCCCACGTGCAGGCCGCGGAGGGGCTCATTCCCGG
GCCCTGATCTCAGAGGCCCGGAATGTGGCTGATAAATCAGAGACTAGACCTGCATG
GCAGGCAGGCCCGACACTCAGCTCCAGGATAAAAGGCCACGGTGTCCCGAGGAGC
CAGGAGGAGCACCCCGCAGGCTGAGGGCAGGTGGGAAGCAAACCCGGACGCATCG
CAGCAGCAGCAGCAGCAGCAGAAGCAGCAGCAGCAGCCTCCGCAGTCCC

14. The method of claim 11, wherein the MTS is present on the 5′ end of the gene of interest.

15. The method of claim 11, wherein the MTS is
(SEQ ID NO: 2)
ATGCAGGCGGCTCGGATGGCCGCGAGCTTGGGGCGGCAGCTGCTGAGGC
TCGGGGGCGGAAGCTCGCGGCTCACGGCGCTCCTGGGGCAGCCCCGGCC
CGGCCCTGCCCGGCGGCCCTATGCCGGG.

16. The method of claim 11, wherein the PH therapeutic is expressed in the right ventricle.

17. The method of claim 11, wherein the PH therapeutic is hydroxyacyl-CoA dehydrogenase trifunctional multienzyme complex subunit alpha (HADHA), Hydroxyacyl-CoA Dehydrogenase Trifunctional Multienzyme Complex Subunit Beta (HADHB), or c-Src.

18. (canceled)

19. The method of claim 11, wherein there is little to no expression of the PH therapeutic in healthy cardiomyocytes.

20. The method of claim 11, wherein the vector is administered intravenously.

21. A method of treating dysfunctional cardiomyocytes in a subject comprising administering a therapeutically effective amount of a vector to the subject,
wherein the vector comprises a nucleic acid sequence comprising a human pro-B-type natriuretic protein (hBNP) promoter operably linked to a mitochondrial targeting sequence (MTS) and/or a gene that encodes a therapeutic,
wherein the therapeutic is expressed in cardiomyocytes undergoing cardiac stress.

22.-47. (canceled)
